SEQUENCE LISTING (70 sequences)

SEQ ID NO: 1 - protein, 883 aa, Artificial Sequence
  Synthetic construct
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - DNA, 18 bp, Artificial Sequence
  Synthetic construct
taatacgactcactatag

SEQ ID NO: 3 - DNA, 18 bp, Artificial Sequence
  Synthetic construct
aattaaccctcactaaag

SEQ ID NO: 4 - DNA, 18 bp, Artificial Sequence
  Synthetic construct
tatttaccctcactaaag

SEQ ID NO: 5 - DNA, 23 bp, Artificial Sequence
  Synthetic construct
  misc_feature (22)..(22): n is a, c, g, or t
atttaggtgacactatagaagng

SEQ ID NO: 6 - protein, 63 aa, Artificial Sequence
  Synthetic construct
ATVKFKYKGEEKEVDISKIKKVWRVGKMISFTYDEGGDKTGRGAVSEKDAPKELLQMLEKQKK

SEQ ID NO: 7 - protein, 88 aa, Pyrococcus furiosus
GRKVRTQQNEILNLLNEKEKAVLRAILEHGGEIKQEDLPELVGYSRPTISKVIQELENKGLIKREKSGKTFVVKIERKIKLDKMGAPT

SEQ ID NO: 8 - protein, 60 aa, Thermotoga maritima
KRRPTINDVAKLAGVSISTVSRYLKDPSQVSEKLGERIREAIKKLGYKPNKIAQGLRTGD

SEQ ID NO: 9 - DNA, 34 bp, Artificial Sequence
  Synthetic construct
cctgcgattgaacacgtgggtcagagagggcagg

SEQ ID NO: 10 - protein, 142 aa, Pyrococcus furiosus
MKTFLTEQQIKVLMLRAKGYKQSEIAKILGTSRANVSILEKRAMEKIEKARNTLLLWEQINSKVIVEIKAGEDIFSIPEKFFKKADKVGVKVPYSTAEIITFLVEHAPVEDRLAKRDFVLFLDSKNKLRIGDCLVIEEIKED

SEQ ID NO: 11 - protein, 75 aa, Pyrococcus furiosus
MPITKVTRNYQITIPAEIRKALGIKEGELLEVRLENGKIIIERLKKERKTLKLGKKLTLEEIEKAIEEGMKQCMQ

SEQ ID NO: 12 - protein, 76 aa, Pyrococcus furiosus
TKIEILRLLKEREMYAYEIWSLLGKPLKYQAVHQHIKELLELGLVEQAYRKGKRVYYKITEKGLRILQNFEDLENI

SEQ ID NO: 13 - protein, 72 aa, Pyrococcus furiosus
MNTGAQGVSEMSRMKIISVQLPQSLIHGLDALVKRGIYPNRSEAIRVAIRELLKKELYKEEIQEEIPEYVVK

SEQ ID NO: 14 - protein, 67 aa, Pyrococcus furiosus
VIIPRPIDPRDIRRIRKELGITQEELARKAGVTQAYIAKLEAGKVDPRLSTFNKILRALIECQKAKI

SEQ ID NO: 15 - protein, 65 aa, Pyrococcus furiosus
NNCECMVVKEKLYTVKQASEILGVHPKTIQKWDREGKIKTVRTPGGRRRIPESEIKRLLGISEEK

SEQ ID NO: 16 - protein, 71 aa, Pyrococcus furiosus
MLKDSAPKRKILEELRKGETVSGDYLASKLGVSRVAIWKHIRELKELGYGIIADKKGYKLVYEPKKPYPWE

SEQ ID NO: 17 - protein, 86 aa, Pyrococcus furiosus
MIDERDKIILEILEKDARTPFTEIAKKLGISETAVRKRVKALEEKGIIEGYTIKINPKKLGYSLVTITGVDTKPEKLFEVAEKLKE

SEQ ID NO: 18 - protein, 77 aa, Pyrococcus furiosus
MEIDDLDRKILSLLIEDSRLSYREIAKKLNVAVGTIYNRIKKLEDMGVIQGFTVKLNYEKLGYELTAIIGIKAQGKK

SEQ ID NO: 19 - protein, 94 aa, Pyrococcus furiosus
EMLWMYILKLLKDRPMYAYEIRNELKKRFGFEPATVSSYVVLYRLEEGGYVSSEWHESEAGRPSRKYYRLTEKGEKLLEKGIETIEDVLNMLKS

SEQ ID NO: 20 - protein, 68 aa, Pyrococcus furiosus
MKVSKATASKVLRSLENKGIVERERRGKTYLVRLTNKGLELLEEISKAGKELDEKIFAEMSVDERIVL

SEQ ID NO: 21 - protein, 56 aa, Pyrococcus furiosus
SEDYMLQNRRKVLAKVLELLNYNPKALNISELARMFGVSRDTIYNDIQQIIKNVEV

SEQ ID NO: 22 - protein, 94 aa, Pyrococcus furiosus
SKEISRFLKVISNPIRYGILKMLNDRWMCVCLISEALEIDQTLVSHHIRILKELDLLEERKEGKLRFYRTNKEKLREYLEKVLEDFNHGTSKGS

SEQ ID NO: 23 - protein, 78 aa, Pyrococcus furiosus
MCRKDVMIISDPKQIKALSDPTRVKILELLRYHPMTVSEISRVIGKDKSTIYRHIKALEEAGLVEEVEKIGNETVYGR

SEQ ID NO: 24 - protein, 122 aa, Pyrococcus furiosus
MEPVEFKLNQKGIKSILPTMEAEIMEYMWEIKEATAGEVYEYMKTKYPEIRRSTVSILMNRLCERGLLKRRMEKGKGGIRYVYSITTTREEFERKVVEKIIESLMMNFREATFAYLSKINKK

SEQ ID NO: 25 - protein, 87 aa, Pyrococcus furiosus
MKKSNLDLLILLAKAGGIEKEILTTSRELSKMLNVSPQTIVRWLEDLEKDGLIKKSESRKGTLVTITEEGVKFLEKLHEELSDALYR

SEQ ID NO: 26 - protein, 104 aa, Unknown
  Thermococcaceae
MEIPPEISHALSEIGFTKYEILTYWTLLVYGPSTAKEISTKSGIPYNRVYDTISSLKLRGFVTEIEGTPKVYAAYSPRIAFFRFKKELEDIMKKLEIELNNVKK

SEQ ID NO: 27 - protein, 75 aa, Pyrococcus furiosus
IINPQARLTPLELEILEIIKQKKSITITEIKEILSERRKSEYPLSLVSEYISRLERKGYVKKIAKGRKKFVEALI

SEQ ID NO: 28 - protein, 94 aa, Pyrococcus furiosus
GIDVVIPEIKHDPIARDIVKILFDLRRANVSQIARELKGRRGKASRNTVRKKLKELEKLGVVKEVPGERGSVYTLSREVVKKWLDLIGIPINLL

SEQ ID NO: 29 - protein, 67 aa, Pyrococcus furiosus
MTKRVKVITDPEVIKVMLEDTRRKILQLLRNREMTISQLSEILGKMPQTIYHHIEKLKEAGLVEVKR

SEQ ID NO: 30 - protein, 100 aa, Pyrococcus furiosus
MEEIKEIMKSHTLGNPVRLGIMIYLFPRRRAPFSHIQKALDLTPGNLDSHIKVLEKHGFVRTYKVIADRPRTMVEITDYGMEETRKFLSHLKTVIDAIHF

SEQ ID NO: 31 - protein, 99 aa, Pyrococcus furiosus
MGEELNRLLDVLGNETRRRILFLLTKRPYFVSELSRELGVGQKAVLEHLRILEEAGLIESRVEKIPRGRPRKYYMIKKGLRLEILLTPTLFGSEMYEAK

SEQ ID NO: 32 - protein, 61 aa, Pyrococcus furiosus
MRRMDKVDLQLIKILSQNSRLTYRELAEMLGTTRQRVARKVDKLKKLGIIRKFTIIPNLEK

SEQ ID NO: 33 - protein, 82 aa, Pyrococcus furiosus
GRKVRTQQNEILNLLNEKEKAVLRAILEHGGEIKQEDLPELVGYSRPTISKVIQELENKGLIKREKSGKTFVVKIERKIKLD

SEQ ID NO: 34 - protein, 59 aa, Thermotoga maritima
KSLQRFLRRNTTSIKHLSEITGVARNRLSDILNGKTQKIRGETLRKIAKAFEKSNILSF

SEQ ID NO: 35 - protein, 107 aa, Thermotoga
DVIQRIKEKYDEFTNAEKKIADTILSDPKGIIESSISDLSEKAGVKSEASVVKFYKKLGLNSFQQFKVLLAQSISRAPLEIVYEDVSSEDDTKTITEKIFKATVRAI

SEQ ID NO: 36 - protein, 101 aa, Thermotoga maritima
KIRDKILNVYTQFSPAERKVADYVLERPDDVIHYSITEFAKIVGVSETTIHRMIKKLDFEGYQAFKIALARELSGLEETIERRDFIDEEIDILRRLKDTLD

SEQ ID NO: 37 - protein, 60 aa, Thermotoga maritima; identical to SEQ ID NO: 8

SEQ ID NO: 38 - protein, 58 aa, Thermotoga maritima
MASIKDVAKLAGVSIATVSRVINGYNNVSEETRKKVIDAIRKLNYHPVYAVKGAVLKR

SEQ ID NO: 39 - protein, 61 aa, Thermotoga maritima
MKKKYVTIRDIAEKAGVSINTVSRALNNKPDISEETRRKILKIAQELGYVKNATASSLRSK

SEQ ID NO: 40 - protein, 58 aa, Thermotoga
MPTIEDVAKLAGVSIATVSRVINGSGYVSEKTRYKVWKAIEELGYKPEISAKLLASKG

SEQ ID NO: 41 - protein, 61 aa, Thermotoga maritima
MRIGEKLRKLRLSRGLTQEELAERTDLSRSFISQLESDKTSPSIDTLERILEALGTDLKHF

SEQ ID NO: 42 - protein, 67 aa, Thermotoga maritima
MHMKTVRQERLKSIVRILERSKEPVSGAQLAEELSVSRQVIVQDIAYLRSLGYNIVATPRGYVLAGG

SEQ ID NO: 43 - protein, 75 aa, Thermotoga maritima
MNTLKKAFEILDFIVKNPGDVSVSEIAEKFNMSVSNAYKYMVVLEEKGFVLRKKDKRYVPGYKLIEYGSFVLRRF

SEQ ID NO: 44 - protein, 81 aa, Thermotoga maritima
MKISKKRRQELIRKIIHEKKISNQFQIVEELKKYGIKAVQPTVARDLKEIGAVKIMDESGNYVYKLLDETPVIDPWKELKR

SEQ ID NO: 45 - protein, 82 aa, Thermotoga maritima
MHKKLNPKSMKRENKKMVLRYLIESGPHSRVEIARKTGLAQSAIWRIIEELVNEGLVEEKGTATGRRRKAVTYGPTRSFITS

SEQ ID NO: 46 - protein, 79 aa, Thermotoga maritima
MPSPLLRRENKIKILRYILKNGKTTRNQLASNLNLAHSTLSYIIDELLDEGFLVFEEIKKKRGRPYQILSVNPEKFTAI

SEQ ID NO: 47 - protein, 79 aa, Thermotoga maritima
MKEERLKEILDIVDRNGFISMKDLQEQLGVSMITVRRDVAELVKRNLVKKVHGGIRKVNYFEKETDFMKRLSINREAKE

SEQ ID NO: 48 - protein, 137 aa, Thermotoga maritima
MFTMRSEYALRLMIVMAKEYGNYLSMTEILEKAKQSVPREFAEKILYTLKKAGLVKTRRGKSGGYMLSRPPKEIKVSEIVFLLDRKSKVFFDMPGCPDELDCVIRALWKRVENEIEKILSGVTLEDLVREQEEKMKQ

SEQ ID NO: 49 - protein, 95 aa, Thermotoga naphthophila
MRDTKGHLKFLVLHIISQQPSHGYYIMKKISQIIGAEPPSPGALYPILSSLRKQKYIETYNEGKRKVYRLTDKGRKYLEEHKEEIKKALDFAERF

SEQ ID NO: 50 - protein, 118 aa, Thermotoga maritima
MRHRGGRGFRGWWLASTILLLVAEKPSHGYELAERLAEFGIEIPGIGHMGNIYRVLADLEESGFLSTEWDTTVSPPRKIYRITPQGKLYLREILRSLEDMKRRIETLEERIKRVLQEE

SEQ ID NO: 51 - protein, 70 aa, Thermotoga maritima
MLSKRDAILKAAVEVFGKKGYDRATTDEIAEKAGVAKGLIFHYFKNKEELYYQAYMSVTEKLQKEFENFL

SEQ ID NO: 52 - protein, 883 aa, Enterobacteria bacteriophage 13a
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSVDNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDTNPDALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEDNHENIMACAKSPLENTWWAEQDSPFCFLAFCFEYAGVQHHGLSYNCSLPLAFDGSCSGIQHFSAMLLDEIGGRAVNLLPSETVQDIYGIVAKKVNVILQADVINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWEAVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLQDILKSDFAFA

SEQ ID NO: 53 - protein, 883 aa, Yersinia bacteriophage YpP-R
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVNLHRQNAGVVGQDSETIELTPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDTNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEDNHENIMACAKSPLENTWWAEQDSPFCFLAFCFEYAGVQHHGLSYNCSLPLAFDGSCSGIQHFSAMLLDEVGGLAVNLLPSATVQDIYGIVAKKVNVILQADVINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWEAVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLQDILKSDFAFA

SEQ ID NO: 54 - protein, 883 aa, Yersinia bacteriophage R
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVNLHRQNAGVVGQDSETIELTPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDTNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEDNHENIMACAKSPLENTWWAEQDSPFCFLAFCFEYAGVQHHGLSYNCSLPLAFDGSCSGIQHFSAMLLDEVGGLAVNLLPSATVQDIYGIVAKKVNVILQADVINGTDNEVVTVTDENTGEIPEKVKLGTKALAGQWLAYGVTRSVTKRSVMTLAYGSKEFGFRQQVLEDTIQPAIDSGKGLMFTQPNQAAGYMAKLIWEAVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLQDILKSDFAFA

SEQ ID NO: 55 - protein, 883 aa, Yersinia bacteriophage phiA1122; identical to SEQ ID NO: 54

SEQ ID NO: 56 - protein, 883 aa, Escherichia bacteriophage CICC 80001
MNTINIAKNDFSDIELAAIPFNTLADHYGERLAREQLALEHESYEMGEARFRKMFERQLKAGEVADNAAAKPLITTLLPKMIARINDWFEVVKAKRGKRPTAFQFLQEIKPEAVAYITIKTTLACLTSADNTTVQAVASAIGRAIEDESRFGRIRDLEAKHFKKNVEEQLNKRVGHVYRKAFMQVVEADMLSKGLMGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDTNPEALTAWKRAAAAVYRKDKARKSRRISLEFMLEQANKFANHKSIWFPYNMDWRGRVYAVSMFNPQGNDMTKGLLTLAKGKPIGKEGYYWLKIHGANCAGVDKVPFPERIKFIEDNHENIMACAKSPLENTWWAEQDSPFCFLAFCFEYAGVQHHGLSYNCSLPLAFDGSCSGIQHFSAMLRDEVGGLAVNLLPSETVQDIYGIVAKKVNVILQEDVINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWEAVSVTVVAAVEAMNWLKSAAKLLAAEVKDKKTGEILRKRCAVHWVTPDGFPVWQEYKKPIQTRLNLIFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLQDILKSDFAFA

SEQ ID NO: 57 - protein, 903 aa, Yersinia bacteriophage YpsP-G
MTERTDGLKKGYMPNGTLYAANRRLVRTWRENNLELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVNLHRQNAGVVGQDSETIELTPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDTNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEDNHENIMACAKSPLENTWWAEQDSPFCFLAFCFEYAGVQHHGLSYNCSLPLAFDGSCSGIQHFSAMLLDEVGGLAVNLLPSATVQDIYGIVAKKVNVILQADVINGTDNEVVTVTDENTGEISEKVKLGTKALAGQWLAYGVTRSVTKRSVMTLAYGSKEFGFRQQVLEDTIQPVIDSGKGLMFTQPNQAAGYMAKLIWEAVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLQDILKSDFAFA

SEQ ID NO: 58 - protein, 883 aa, Salmonella bacteriophage Vi06
MNTISITKNDFSDIELAAIPFNTLADHYGERLAREQLALEHESYEMGEVRFRKMFERQLKAGEIADNDATKPLITTLLPKMIARINSWFKEVQAKCGKRPTAFQFLQGIKPEAIAYITIKTTLARLTSMDNTTVQAVASAIGRAIEDEARFGRIRDLEAKHFKKNVEEQLNKRVGHVYKKAFMQVIEADMLSKGLLGGESWSSWHKEDSIHVGVRCIEMLIESTGMVSLHRQNAGVVGQDSETIELAPEYAEAIATRAGALAGISPMFQPCVVPPKPWTSISGGGYWANGRRPLALVRTHSKKALMRYADVYMPEVYKAVNIAQNTAWRINKKVLAVANVVTKWKHCPVDYIPTIEREELPMKPEDIDTNPEALASWKRAAAAVYRKDKARKSRRMSLEFMLEQANKFANHRAIWFPYNMDWRGRVYAVSMFNPQGNDMTKGLLTLAKGKPIGKEGFYWLKIHGANCAGVDKVPFPERIKFIEDNHENILACAKSPLENTWWSEQDSPFCFLAFCFEYAGGQHHGLSYNCSLPLAFDGSCFGIQHFSVMLRDEVGGRAVNLLPSETVQDIYGIVAKKVNEILQVDMINGTDNEVVTVTDDKTGEIYEKIKLGTKELAGQWLAYGVTRSVTKRSVMTLAYGSKEFGFRQQVLEDTIQPAIDSGKGLMFTHPNQAAGYMAKLIWEAVSVTVVAAVEAMNWLKSAAKLLAVEVKDRKTGEILRKRCAVHWTTPDGFPVWQEYKKPVQTRLNLIFLGQFRLQPTINTNRDSEIDAYKQESGIAPNFVHSQDGSHLRKTVVWAHEKYGIDSFALIHDSFGTIPADAANLFKAVRETMVATYESCDVLADFYAQFADQLHKSQLDKMPVLPSKGNLNLQDILKSDFAFA

SEQ ID NO: 59 - protein, 883 aa, Stenotrophomonas bacteriophage IME15
MTVIAIEKNDFSDVELAVIPFNTLADHYGEKLAREQLALEHEAYEMGEARFRKIFERQLKAGEVADNAAAKPLVATLLPKMIERIHAWFEEVSAKRGKRPTAFKFLQEVKPEAIAYITIKTVLGTLTSAEQTTVQAAASAVGRAIEDEARFGRIRDLEAKHFKKNVEEQLNKRVGHVYKKAFLQVVEADMLSKGLMGGEAWSSWHKEDSIHVGVRCIEMLIEATGLVVLERQNAGVVGADAETLSLASEYADAIATRAGALAGISPMYQPCVVPPKPWTTVTGGGYWANGRRPLALVRTHGKKALMRYEDVYMPEVYKAVNLAQSTAWKINKKVLAVANEITKWKHCPVEDIPAIEREELPVKPDDIDENPEALTNWKRAAAAVYRKDKARKSRRLSLEFMLEQANKFANHKAIWFPYNMDWRGRVYAVSMFNPQGNDMTKGLLTLAKGKAIGKEGFYWLKIHGANCAGVDKVPFPERIKFIEDNHEHIMASAKNPLEYTWWAEQDSPFCFLAFCFEYAGVMHHGLSYNCSLPLAFDGSCSGIQHFSAMLRDEVGGRAVNLLPSETVQDIYGIVAKKVNEIMQRDVISGTDDELVTETDKTTGEITEKAVLGTRTLAGQWLAYGANRSVTKRSVMTLAYGSKEFGFRQQVLEDTIRPAIDSGKGLMFTIPNQAAGYMAKLIWDSVSVTVVAAVEAMKWLQSAAKLLAAEVKDKKTGEVLRNRCAVHWVTPDGFPVWQEYRKPLQTRLNLMFLGQFRLQPTINTNKDSGIDAHKQESGIAPNFVHSQDGSHLRKTVVWAHEKYGIESFALIHDSFGTIPADAGNLFKAVRETMVDTYENCDVLADFYEQFADQLHESQLDKMPALPKKGNLNLRDILESDFAFA

SEQ ID NO: 60 - protein, 884 aa, Citrobacter bacteriophage SH2
MNIIENIEKNDFSEIELAAIPFNTLADHYGSALAREQLALEHESYELGERRFLKMLERQAKAGEIADNAAAKPLLATLLPKLTARIVEWLEEYASKKGRKPVAYAPLQLLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVSITGGGYWANGRRPLALVRTHSKKGLMRYEDVYMPEVYKAVNIAQNTAWKINKKVLAVVNEIVNWKNCPVADIPSLERQELPPKPDDIDTNEAALKEWKKAAAGVYRLDKARVSRRISLEFMLEQANKFANKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVAHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVITYENNDVLADFYDQFADQLHETQLDKMPPLPKKGNLNLQDILKSDFAFA

SEQ ID NO: 61 - protein, 884 aa, Enterobacter bacteriophage E-4
MNIIENIEKNDFSEIELAAIPFNTLADHYGSALAREQLALEHESYELGERRFLKMLERQAKAGEIADNAAAKPLLATLLPKLTARIVEWLEEYASKKGRKPSAYAPLQLLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVSITGGGYWANGRRPLALVRTHSKKGLMRYEDVYMPEVYKAVNIAQNTAWKINKKVLAVVNEIVNWKNCPVADIPSLERQELPPKPDDIDTNEAALKEWKKAAAGIYRLDKARVSRRISLEFMLEQANKFANKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVAHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVLTYENNDVLADFYDQFADQLHETQLDKMPPLPKKGNLNLQDILKSDFAFA

SEQ ID NO: 62 - protein, 884 aa, Yersinia bacteriophage phiYe-F10
MNIIENIEKNDFSEIELAAIPFNTLADHYGSALAREQLALEHESYELGERRFLKMLERQAKAGEIADNAAAKPLLATLLPKLTARIVEWLEEYASKKGRKPVAYAPLQSLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVSITGGGYWANGRRPLALIRTHSKKGLMRYEDVYMPEVYKAVNIAQNTAWKINKKVLAVVNEIVNWKNCPVADIPSLERQELPPKPDDIDTNEAALKEWKKAAAGVYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVAHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVITYENNDVLADFYDQFADQLHETQLDKMPPLPKKGNLNLQDILKSDFAFA

SEQ ID NO: 63 - protein, 884 aa, Citrobacter bacteriophage phiCFP-1
MNIIENIEKNDFSEIELAAIPFNTLADHYGSALAREQLALEHESYELGERRFLKMLERQAKAGEIADNAAAKPLLATLLPKLTARIVEWLEEYDSKKGRKPVAYAPLQLLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNIAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVAHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVITYENNDVLADFYDQFADQLHETQLDKMPPLPKKGNLNLQDILKSDFAFA

SEQ ID NO: 64 - protein, 884 aa, Citrobacter bacteriophage SH1
MNIIENIEKNDFSEIELAAIPFNTLADHYGSALAREQLALEHESYELGERRFLKMLERQAKAGEIADNAAAKPLLATLLPKLTARIVEWLEEYASKKGRKPVAYAPLQLLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVSITGGGYWANGRRPLALVRTHSKKGLMRYEDVYMPEVYKAVNIAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVAHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLADEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVITYENNDVLADFYDQFADQLHETQLDKMPPLPKKGNLNLQDILKSDFAFA

SEQ ID NO: 65 - protein, 884 aa, Salmonella bacteriophage phiSG-JL2
MNIIENIEKNDFSEIELAAIPFNTLADHYGSALAREQLALEHESYELGERRFLKMLERQAKAGEIADNAAAKPLLATLLPKLTARIVEWLEEYASKKGRKPVAYAPLQLLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNIAQNTAWKINKKVLAVVNEIVNWKNCPVADIPSLERQELPPKPDDIDTNEAALKEWKKAAAGVYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVAHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVLTYENNDVLADFYDQFADQLHETQLDKMPPLPKKGKLNLQDILKSDFAFA

SEQ ID NO: 66 - protein, 884 aa, Yersinia bacteriophage phiYeO3-12
MNIIENIEKNDFSEIELAAIPFNTLADHYGSALAREQLALEHESYELGERRFLKMLERQAKAGEIADNAAAKPLLATLLPKLTTRIVEWLEEYATKKGRKPVAYAPLQSLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNIAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVAHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHENYGIESFALIHDSFGTIPADAGKLFKAVRETMVITYENNDVLADFYDQFADQLHETQLDKMPPLPKKGNLNLQDILKSDFAFA

SEQ ID NO: 67 - protein, 884 aa, Enterobacteria bacteriophage T7M
MNIIENIEKNDFSEIELAAIPFNTLADHYGSALAKEQLALEHESYELGERRFLKMLERQAKAGEIADNAAAKPLLATLLPKLTTRIVEWLEEYASKKGRKPSAYAPLQLLKPEASAFITLKVILASLTSTNMTTIQAAAGMLGKAIEDEARFGRIRDLEAKHFKKHVEEQLNKRHGQVYKKAFMQVVEADMIGRGLLGGEAWSSWDKETTMHVGIRLIEMLIESTGLVELQRHNAGNAGSDHEALQLAQEYVDVLAKRAGALAGISPMFQPCVVPPKPWVAITGGGYWANGRRPLALVRTHSKKGLMRYEDVYMPEVYKAVNLAQNTAWKINKKVLAVVNEIVNWKNCPVADIPSLERQELPPKPDDIDTNEAALKEWKKAAAGIYRLDKARVSRRISLEFMLEQANKFASKKAIWFPYNMDWRGRVYAVPMFNPQGNDMTKGLLTLAKGKPIGEEGFYWLKIHGANCAGVDKVPFPERIAFIEKHVDDILACAKDPINNTWWAEQDSPFCFLAFCFEYAGVTHHGLSYNCSLPLAFDGSCSGIQHFSAMLRDEVGGRAVNLLPSETVQDIYGIVAQKVNEILKQDAINGTPNEMITVTDKDTGEISEKLKLGTSTLAQQWLAYGVTRSVTKRSVMTLAYGSKEFGFRQQVLDDTIQPAIDSGKGLMFTQPNQAAGYMAKLIWDAVSVTVVAAVEAMNWLKSAAKLLAAEVKDKKTKEILRHRCAVHWTTPDGFPVWQEYRKPLQKRLDMIFLGQFRLQPTINTLKDSGIDAHKQESGIAPNFVHSQDGSHLRMTVVYAHEKYGIESFALIHDSFGTIPADAGKLFKAVRETMVITYENNDVLADFYSQFADQLHETQLDKMPPLPKKGNLNLQDILKSDFAFA

SEQ ID NO: 68 - protein, 884 aa, Enterobacteria bacteriophage T3; identical to SEQ ID NO: 67

SEQ ID NO: 69 - protein, 883 aa, Artificial Sequence
  Synthetic construct
MNIINIAKNDFSDIELAAIPFNILADHYGAQLAREQLALEHEAYEEGEKRFLKMLERQIKAGEFADNAAAKPLLSTLLPKLIARINDWFEEVAAKRGKKPVAYNPLQHVKPEAAAFITLKVTLACLTKAEFTTIQAVASAIGRAIEDEARFGRIRDLEAKHFKKHVEEQLNKRVGHVYKKAFMQVVEADMLSKGLLGGEAWSSWTKEESIHVGVRMLELLIESTGLVELHRPNAGNVGKDVEMIQLAPEYVDLLAKRAGALAGISPMYQPCVVPPKPWTSIVGGGYWANGRRPLALVRTHSKKALMRYEDVYMPEVYKAVNIAQNTPWKINKKVLAVVNEIVNWKHCPVADVPAIEREELPPKPEDIDTNEAALKAWKKAAAAIYRKDKARVSRRLSMEFMLEQANKFANFKAIWFPYNMDWRGRVYAVPMFNPQGNDMTKGLLTLAKGKPIGKDGFYWLKIHGANCAGVDKVPFPERIKFIEDNHENIMACAKDPLNNEWWAEQDSPFCFLAFCFEYAGVQHHGLSYNCSLPLAFDGSCSGIQHFSAMLRDEIGGRAVNLLPSETVQDIYGIVADKVNEILKQDAINGTDNEVETVTDKDTGEITEKLKLGTKELAGQWLAYGVTRKVTKRSVMTLAYGSKEFGFRQQVLEDTIQPAIDSGKGLMFTQPNQAAGYMAKLIWEAVSVTVVAAVEAMNWLKSAAKLLAAEVKDKKTKEVLRKRCAVHWVTPDGFPVWQEYRKPVQTRLNLMFLGQFRLQPTINTNKDSEIDAHKQESGIAPNFVHSQDGSHLRMTVVHAHEKYGIESFALIHDSFGTIPADAGNLFKAVRETMVNTYEDNDVLADFYDQFADQLHESQLDKMPALPAKGNLNLQDILKSDFAFA

SEQ ID NO: 70 - protein, 883 aa, Artificial Sequence
  Synthetic construct
MNTINIAKNDFSDIELAAIPFNTLADHYGERLAREQLALEHESYEMGEKRFLKMLERQVKAGEIADNAAAKPLITTLLPKLTARINDWFEEVAAKRGKRPVAYQPLQGIKPEAVAFITIKVVLASLTSADNTTIQAVASAIGRAIEDEARFGRIRDLEAKHFKKHVEEQLNKRVGHVYKKAFMQVVEADMLSKGLLGGEAWSSWNKEESMHVGIRMIEMLIESTGLVELHRHNAGVVGQDSETIQLAPEYVEALAKRAGALAGISPMFQPCVVPPKPWVSITGGGYWANGRRPLALVRTHSKKALMRYEDVYMPEVYKAVNIAQNTAWKINKKVLAVVNEIVNWKHCPVEDIPAIEREELPPKPDDIDTNEEALKAWKKAAAAVYRKDKARKSRRISLEFMLEQANKFANHKAIWFPYNMDWRGRVYAVPMFNPQGNDMTKGLLTLAKGKPIGKEGFYWLKIHGANCAGVDKVPFPERIKFIEDNHDNIMACAKDPLDNTWWAEQDSPFCFLAFCFEYAGVQHHGLSYNCSLPLAFDGSCSGIQHFSAMLRDEVGGRAVNLLPSETVQDIYGIVADKVNEILKQDVINGTDNEVVTVTDKDTGEISEKLKLGTKELAQQWLAYGVTRSVTKRSVMTLAYGSKEFGFRQQVLEDTIQPAIDSGKGLMFTQPNQAAGYMAKLIWDAVSVTVVAAVEAMNWLKSAAKLLAAEVKDKKTKEILRKRCAVHWVTPDGFPVWQEYRKPIQTRLNLMFLGQFRLQPTINTNKDSEIDAHKQESGIAPNFVHSQDGSHLRMTVVYAHEKYGIESFALIHDSFGTIPADAGNLFKAVRETMVNTYENNDVLADFYDQFADQLHESQLDKMPALPAKGNLNLQDILKSDFAFA

(19) United States
(12) Patent Application Publication
(10) Pub. No.: US 2019/0024061 A1
(43) Pub. Date: Jan. 24, 2019

(54) Thermostable Variants of T7 RNA Polymerase

(71) Applicants: Jennifer ONG, Salem, MA (US); Shaorong CHONG, Somerville, MA (US); Haruichi ASAHARA, Boxford, MA (US); Kuo-Chan HUNG, Beverly, MA (US); Vladimir PATAPOV, Auburndale, MA (US); George TZERTZINIS, Cambridge, MA (US); New England Biolabs, Inc., Ipswich, MA (US)

(72) Inventors: Jennifer Ong, Salem, MA (US); Shaorong Chong, Ipswich, MA (US); Haruichi Asahara, Ipswich, MA (US); Kuo-Chan Hung, Ipswich, MA (US); Vladimir Potapov, Auburndale, MA (US); George Tzertzinis, Ipswich, MA (US)

(73) Assignee: New England Biolabs, Inc., Ipswich, MA (US)

(21) Appl. No.: 16/068,020

(22) PCT Filed: Jan. 12, 2017
(86) PCT No.: PCT/US2017/013179

§ 371 Date: Jul. 3, 2018

Related U.S. Application Data
(60) Provisional application No. 62/278,161, filed on Jan. 13, 2016.
(60) Provisional application No. 62/278,161, filed on Jan. 13, 2016.

(51) Int. Cl. C12N 9/12; C12P 19/34

(52) U.S. Cl. CPC C12N 9/1247; C12P 19/34; C12Y 207/07006

(57) ABSTRACT

A bacteriophage RNA polymerase variant is provided. In some embodiments, the variant may have increased thermostability relative to the corresponding wild type bacteriophage RNA polymerase and/or wild type T7 RNA polymerase. Compositions, kits and methods that employ the variant are also provided.

### CROSS-REFERENCE

This application is a § 371 application of international application number PCT/US2017/013179 filed Jan. 12, 2017, which claims priority from U.S. provisional application No. 62/278,161 filed Jan. 13, 2016 and U.S. provisional application No. 62/416,770 filed Nov. 3, 2016, herein incorporated by reference.

### BACKGROUND

Thermostable and thermoactive enzymes have great utility in academic research and industrial applications. The high stability of enzymes from thermophilic organisms enables technologies in molecular biology and diagnostics (the Polymerase Chain Reaction, for example). However, equivalent enzymes from thermophilic organisms are not always available. In these cases, directed evolution or computational methods can serve as a powerful tool to identify variants of mesophilic enzymes that confer thermostability. For example, current in vitro transcription methods are limited to reaction temperatures below 45° C. The typical viral RNA polymerases that carry out these reactions are not active at elevated temperatures, and there is a need to identify thermoactive and stable variants in order to carry out in vitro transcription reactions at elevated temperatures.

### SUMMARY

A bacteriophage RNA polymerase variant is provided. In some embodiments, the variant may have increased thermostability and/or activity at elevated reaction temperatures relative to a corresponding wild type RNA polymerase. Compositions, kits and methods that employ the variant are also provided.

In some embodiments, the variant: (i) comprises an amino acid sequence that has at least 80% (e.g., at least 90%, or at least 95%) sequence identity to SEQ ID NO:1; and (ii) comprises an amino acid substitution at one or more positions corresponding to positions 109, 205, 388, 534, 567 and 618 of SEQ ID NO:1. In some embodiments, the variant may comprise an amino acid substitution at least two positions corresponding to positions 109, 205, 388, 534, 567 and 618 of SEQ ID NO:1. In some embodiments, the variant may comprise an amino acid substitution at least three positions corresponding to positions 109, 205, 388, 534, 567 and 618 of SEQ ID NO:1. In some embodiments, the variant may comprise an amino acid substitution at positions corresponding to positions 109, 205, 388, 534, 567 and 618 of SEQ ID NO:1. For example, in some embodiments, the variant may comprise one or more of the following amino acids substitutions: I109L, H205S, D388E, L534V, V567P and G618Q wherein the amino acid substitutions are at positions that correspond to positions in SEQ ID NO:1.

In one example, variant further includes an amino acid substitution at one or more positions corresponding to positions: 75, 83, 108, 206, 227, 281, 297, 312, 323, 327, 333, 340, 354, 362, 375, 428, 446, 454, 461, 495, 510, 584, 591, 642, 711, 724, 740, 788, 832, 834, 835, 843, 847, 849, 856, 863, 866 and 877 of SEQ ID NO:1.

In another example, the variant may further comprise an amino acid substitution of at least 10 positions corresponding to positions: 75, 83, 108, 206, 227, 281, 297, 312, 323, 327, 333, 340, 354, 362, 375, 428, 446, 454, 461, 495, 510, 584, 591, 642, 711, 724, 740, 788, 832, 834, 835, 843, 847, 849, 856, 863, 866 and 877 of SEQ ID NO:1.

In another example, the variant may further comprise one or more of the following amino acids substitutions: T75Q, A83K, E108L, K206P, V227I, I281P, V297I, Y312D, A323I, A327P, K333P, V340E, A354Q, M362P, T375K, T375N, A428P, L446F, K454P, K461R, S495N, C510Q, A584K, D591E, K642R, K711R, A724P, K740R, G788A, M832F, D834E, T835L, A843Q, D847E, F849V, 5856T, A863P, A866K and E877R, wherein the amino acid substitutions are at positions that correspond to positions in SEQ ID NO:1.

In another example, the isolated bacteriophage RNA polymerase variant, wherein the variant includes at least 10 of the following amino acids substitutions: T75Q, A83K, E108L, K206P, V227I, I281P, V297I, Y312D, A323I, A327P, K333P, V340E, A354Q, M362P, T375K, T375N, A428P, L446F, K454P, K461R, S495N, C510Q, A584K, D591E, K642R, K711R, A724P, K740R, G788A, M832F, D834E, T835L, A843Q, D847E, F849V, 5856T, A863P, A866K, and E877R, wherein the amino acid substitutions are at positions that correspond to positions in SEQ ID NO:1.

In some embodiments, any isolated bacteriophage RNA polymerase variant described above may include a fusion to an exogenous DNA binding domain. Examples are provided in Table 1. In another embodiment, the variant has increased stability at temperatures of at least 45° C. (e.g., at or above 50° C., or at or above 55° C.) relative the T7 RNA polymerase of SEQ ID NO:1 as a result of the one or more amino acid substitutions.

Also provided is a composition that includes i. an isolated bacteriophage RNA polymerase variant described above; and ii. a buffering agent. The composition may further include ribonucleoside triphosphates and/or modified nucleotides. The composition may further include a template DNA molecule comprising: a bacteriophage promoter (e.g., a T7 or T3 RNA polymerase promoter) operably linked to a target nucleotide sequence to be transcribed.

Also provided is a kit is provided that includes i. an isolated bacteriophage RNA polymerase variant of any of those described above; and ii. a reaction buffer. The kit may further comprise one or more ribonucleoside triphosphates and/or modified nucleotides.

Also provided is a method is provided for synthesizing an RNA molecule that includes (a) combining an isolated bacteriophage RNA polymerase variant described above with ribonucleoside triphosphates and/or modified nucleotides and a template DNA molecule comprising a bacteriophage RNA promoter that is operably linked to a target nucleotide sequence to be transcribed, to produce a reaction mix; and (b) incubating the reaction mix to transcribe the template DNA molecule into RNA.

In one aspect, the incubating is done at a temperature of at least 45° C. or at above 50° C. or at above 55° C. (for example 45° C. to 60° C., 45° C. to 50° C., 50° C. to 55° C. or 55° C. or 60° C.).

One example of a bacteriophage RNA polymerase is T7 RNA polymerase.

### BRIEF DESCRIPTION OF THE DRAWINGS

The skilled artisan will understand that the drawings, described below, are for illustration purposes only. The drawings are not intended to limit the scope of the present teachings in any way.

FIGS. 1A-1D are graphs showing the effect of various amino acid substitutions on the activity of T7 RNA polymerase (SEQ ID NO: 1). FIG. 1A-1C show data for selected variants that have individual mutations. These reactions were done at 45° C. for 10 hours (FIG. 1A and FIG. 1B) and at 37° C. for 2 hours followed by 45° C. for 8 hours (FIG. 1C). FIG. 1D shows the additive effect of individual mutations identified by Tth PURE assay. The reaction was carried out at 45° C. for 10 hours. The thermostability of variants was estimated using the formula, (M−WT)/WT, in which M and WT stand for the maximum value of fluorescent signal from synthesized GFP in 10-hour reactions with mRNA of T7 RNA polymerase variant and wild-type, respectively. In this assay, if a variant polymerase has an activity of “0” then it has the equivalent activity as the wild type T7 RNA polymerase. If a variant polymerase has an activity of “0.5” then it has a 50% increase in activity relative to the wild type T7 RNA polymerase.

FIGS. 1A-1C show results for 45 single amino acid variants of T7 RNA polymerase.

FIG. 1D shows the additive effect of combining amino acid substitutions. In FIG. 1D, the additive effect of 1, 2, 3, 4, 5 and 6 amino acids substitutions is shown.

FIG. 2 shows the melting temperature of wild type T7 RNA polymerase (WT), as well various variants of the same (i.e., M1, M2, M3b, M4, M5, etc.). In this graph, the number after the M corresponds to the number of amino acid substitutions in the polymerase. For example, M5 has five amino acid substitutions relative to wild type T7 RNA polymerase, etc. This data shows that the effect of amino acid substitutions on melting temperature is largely additive.

FIGS. 3A-3C show that (i) mutant RNA polymerases at temperatures above about 45° C. make more RNA compared to the corresponding wild type; (ii) mutant RNA polymerases at temperatures in the range of 50° C. to 55° C. can make at least 2 fold more RNA than the corresponding wild type RNA polymerase; and (iii) fusion proteins containing RNA polymerase and a DNA binding domain are more active at high temperatures than the corresponding wild type RNA polymerase; and (iv) fusion proteins containing RNA polymerase and a DNA binding domain show prolonged activity with a more gradual loss of activity at temperatures above 56° C. compared to the same RNA polymerase variant alone.

FIG. 3A shows the transcription activity (RNA synthesis yield) at increased temperature for the wild type T7 RNA polymerase, as well as two variants, commercial T7 RNA polymerase from Toyobo “Toyobo” and M20 where M20 is a mutant of T7 RNA polymerase with 20 amino acid substitutions. As shown, the M20 variant is highly active at temperatures at above 55° C.

FIG. 3B is a graph showing the transcription activity of wild type T7 polymerase compared to the activity of a variant M18 and a variant M13 and a fusion protein containing the M18 variant and the DNA binding domain of a lacl-like protein from Thermotoga (007) after a 20 minute incubation. As shown the M13 and M18 variants and also the M18 fusion protein is highly active at temperatures at above 55° C. with the fusion protein maintaining its activity (Fluorescence units on the Y axis corresponds to amount of RNA).

FIG. 3C shows a comparison between wild type, mutant and fusion between mutant and DNA binding domain of a thermostable protein (see Table 1) in which not only is the activity of the fusion variant higher at increasing temperature than the variant alone but also there is slower reduction of activity at temperatures about 56° C. compared to the wild type.

FIG. 4 is a graph showing the transcription activity of wild type T7 RNA polymerase at different temperatures, compared to the transcription activity of the same T7 RNA polymerase fused to the SS07 DNA binding domain (SS07-T7), the DNA binding domain from a helix-turn-helix (HTH) from Pyrococcus furiosus (109-T7) and the DNA binding domain of a lacl-like protein from Thermotoga (007-T7). The fusion proteins containing T7 RNA polymerase and each of the three DNA binding domain are more active at high temperatures than wild type T7 RNA polymerase.

FIG. 5 is a graph showing the transcription activity of wild type T7 polymerase compared to the activity of variant M18 which contains 18 amino acid substitutions and a fusion protein containing the M18 variant and the DNA binding domain of a lacl-like protein from Thermotoga (007). Fusion proteins containing thermostable T7 RNA polymerase variants and a DNA binding domain are more active at high temperatures than the thermostable T7 RNA polymerase variants.

FIG. 6 shows improved thermostability for the T7 RNA polymerase variant identified in SEQ ID NO:70 at 50° C. compared with T7 RNA polymerase wild type under the same reaction conditions

### DEFINITIONS

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

The term “non-naturally occurring” refers to a composition that does not exist in nature.

In the context of a protein, the term “non-naturally occurring” refers to a protein that has an amino acid sequence and/or a post-translational modification pattern that is different to the protein in its natural state. For example, a non-naturally occurring protein may have one or more amino acid substitutions, deletions or insertions at the N-terminus, the C-terminus and/or between the N- and C-termini of the protein. A “non-naturally occurring” protein may have an amino acid sequence that is different to a naturally occurring amino acid sequence but that that is at least 80%, at least 85%, at least 90%, at least 95%, at least 97%, at least 98% or at least 99% identical to a naturally occurring amino acid sequence. In certain cases, a non-naturally occurring protein may contain an N-terminal methionine or may lack one or more post-translational modifications (e.g., glycosylation, phosphorylation, etc.) if it is produced by a different (e.g., bacterial) cell.

In the context of a nucleic acid, the term “non-naturally occurring” refers to a nucleic acid that contains: a) a sequence of nucleotides that is different to a nucleic acid in its natural state, b) one or more non-naturally occurring nucleotide monomers (which may result in a non-natural backbone or sugar that is not G, A, T or C) and/or C) may contain one or more other modifications (e.g., an added label or other moiety) to the 5′-end, the 3′ end, and/or between the 5′- and 3′-ends of the nucleic acid.

In the context of a preparation, the term “non-naturally occurring” refers to: a) a combination of components that are not combined by nature, e.g., because they are at different locations, in different cells or different cell compartments; b) a combination of components that have relative concentrations that are not found in nature; c) a combination that lacks something that is usually associated with one of the components in nature; e) a combination that is in a form that not found in nature, e.g., dried, freeze dried, crystalline, aqueous; and/or d) a combination that contains a component that is not found in nature. For example, a preparation may contain a buffering agent (e.g., Tris, HEPES, TAPS, MOPS, tricine or MES), a detergent, a dye, a reaction enhancer or inhibitor, an oxidizing agent, a reducing agent, a solvent or a preservative that is not found in nature.

As used herein, the term “buffering agent”, refers to an agent that allows a solution to resist changes in pH when acid or alkali is added to the solution. Examples of suitable non-naturally occurring buffering agents that may be used in the compositions, kits, and methods of the invention include, for example, Tris, HEPES, TAPS, MOPS, tricine, or MES.

The term “corresponding to” in the context of corresponding positions, refers to positions that lie across from one another when sequences are aligned, e.g., by the BLAST algorithm.

The term “variant T7 RNA polymerase” may encompass other types of bacteriophage RNA polymerase with sequences of at least 80% identity to wild type T7 RNA polymerase (SEQ ID NO:1). Enzymes having a similar architecture can be identified using the Conserved Domain Architecture Retrieval Tool (CDART) program of the National Center for Biotechnology Information (Geer, et al. Genome Research 12:1619-1623 (2002)) or by other predictive programs, based on searches employing the sequence of T7 RNA polymerase. Examples of enzymes identified in this manner include: T odd bacteriophages or related viruses including Enterobacteria bacteriophage T7, Yersinia pestis bacteriophage phiA1122; Pseudomonas bacteriophage gh-1; bacteriophage of Pseudomonas putida; Bacteriophage T3; Roseophage SIO1; and Bacteriophage phiYeO3-12. In addition other related bacteriophages such as SP6, bacteriophage phiKMV, Enterobacteria bacteriophage K1-5, Vibriophage VpV262, BA14, BA127 and BA156 may encode similar enzymes.

The term “fusion protein” refers to a DNA binding domain linked to a wild type or variant polymerase. Examples include Pyrococcus furiosus (109-T7) and the DNA binding domain of a lacl-like protein from Thermotoga (007-T7). Other examples are listed in Table 1.

TABLE 1
DNA binding proteins

| | | | |
|---|---|---|---|
| DNA-binding protein Tfx | BD-51 | gi\|499321160 | SEQ ID NO: 10 |
| AbrB/MazE/MraZ-like | BD-52 | gi\|499321199 | SEQ ID NO: 11 |
| “Winged helix” DNA-binding domain | BD-54 | gi\|499322061 | SEQ ID NO: 12 |
| Ribbon-helix-helix protein, copG family | BD-62 | gi\|499321149 | SEQ ID NO: 13 |
| lambda repressor-like DNA-binding domains | BD-63 | gi\|499322443 | SEQ ID NO: 14 |
| Resolvase-like | BD-67 | gi\|499322676 | SEQ ID NO: 15 |
| “Winged helix” DNA-binding domain | BD-71 | gi\|499322676 | SEQ ID NO: 16 |
| “Winged helix” DNA-binding domain | BD-74 | gi\|499322255 | SEQ ID NO: 17 |
| “Winged helix” DNA-binding domain | BD-75 | gi\|499322388 | SEQ ID NO: 18 |
| “Winged helix” DNA-binding domain | BD-81 | gi\|499322131 | SEQ ID NO: 19 |
| “Winged helix” DNA-binding domain | BD-82 | gi\|499321342 | SEQ ID NO: 20 |
| “Winged helix” DNA-binding domain | BD-85 | gi\|499321130 | SEQ ID NO: 21 |
| “Winged helix” DNA-binding domain | BD-86 | gi\|499322705 | SEQ ID NO: 22 |
| “Winged helix” DNA-binding domain | BD-88 | gi\|499320855 | SEQ ID NO: 23 |
| “Winged helix” DNA-binding domain | BD-89 | gi\|499322250 | SEQ ID NO: 24 |
| “Winged helix” DNA-binding domain | BD-91 | gi\|499321633 | SEQ ID NO: 25 |
| “Winged helix” DNA-binding domain | BD-92 | gi\|490170077 | SEQ ID NO: 26 |
| “Winged helix” DNA-binding domain | BD-93 | gi\|499321272 | SEQ ID NO: 27 |
| “Winged helix” DNA-binding domain | BD-94 | gi\|499320919 | SEQ ID NO: 28 |
| “Winged helix” DNA-binding domain | BD-97 | gi\|499320853 | SEQ ID NO: 29 |
| “Winged helix” DNA-binding domain | BD-98 | gi\|499321734 | SEQ ID NO: 30 |
| “Winged helix” DNA-binding domain | BD-100 | gi\|499322439 | SEQ ID NO: 31 |
| “Winged helix” DNA-binding domain | BD-102 | gi\|499322707 | SEQ ID NO: 32 |
| “Winged helix” DNA-binding domain | BD-109 | gi\|499321112 | SEQ ID NO: 33 |
| HCP-like | BD-02 | gi\|351675391 | SEQ ID NO: 34 |
| Helix-turn-helix domain, rpiR family | BD-03 | gi\|500479591 | SEQ ID NO: 35 |
| Helix-turn-helix domain, rpiR family | BD-04 | gi\|15643984 | SEQ ID NO: 36 |
| Bacterial regulatory proteins, lacl family | BD-07 | gi\|15643711 | SEQ ID NO: 37 |
| Bacterial regulatory proteins, lacl family | BD-08 | gi\|15643974 | SEQ ID NO: 38 |
| Bacterial regulatory proteins, lacl family | BD-09 | gi\|15643956 | SEQ ID NO: 39 |
| Bacterial regulatory proteins, lacl family | BD-11 | gi\|500480095 | SEQ ID NO: 40 |
| lambda repressor-like DNA-binding domains | BD-12 | gi\|15643421 | SEQ ID NO: 41 |
| “Winged helix” DNA-binding domain | BD-14 | gi\|15644350 | SEQ ID NO: 42 |
| “Winged helix” DNA-binding domain | BD-16 | gi\|24159093 | SEQ ID NO: 43 |
| “Winged helix” DNA-binding domain | BD-18 | gi\|15643139 | SEQ ID NO: 44 |
| “Winged helix” DNA-binding domain | BD-23 | gi\|15642807 | SEQ ID NO: 45 |
| “Winged helix” DNA-binding domain | BD-24 | gi\|15643159 | SEQ ID NO: 46 |
| “Winged helix” DNA-binding domain | BD-30 | gi\|15643333 | SEQ ID NO: 47 |
| “Winged helix” DNA-binding domain | BD-32 | gi\|15643055 | SEQ ID NO: 48 |
| “Winged helix” DNA-binding domain | BD-37 | gi\|15643827 | SEQ ID NO: 49 |
| “Winged helix” DNA-binding domain | BD-43 | gi\|15643699 | SEQ ID NO: 50 |
| Homeodomain-like | BD-45 | gi\|15643788 | SEQ ID NO: 51 |

The term “temperature-sensitive inhibitor” includes antibody-based hot start RNA polymerase inhibitors where examples of hot start inhibitors for polymerases is provided in Kellogg, et al., Biotechniques, 16(6):1134-7 (1994); aptamer based hot start RNA polymerases where examples for polymerases are provided by New England Biolabs, Ipswich, Mass. (catalog # M0495) and also described by Dang, et al., Journal of Molecular Biology, 264(2), 268-278 (1996); Affibody-based hot start inhibitors where Affibody is a protein-based ligand that inhibits DNA polymerase and exonuclease activity at low temperatures but not at higher temperatures (also described by (Thermo Fisher Scientific, Waltham, Mass. catalog # F549L); and chemical modification resulted in hot start RNA polymerase (see for example U.S. Pat. No. 5,773,258; and U.S. Pat. No. 6,183,998).

The term “promoter sequence” includes the sequence 5′-TAATACGACTCACTATAG-3′ and any sequence that is at least 90% identical to the canonical sequences for T7. See also Dunn, et al., J Mol Biol. 166(4):477-535 (1983) and Ikeda, et al., J. Biol. Chem. 26, (16): 11322-11328 (1992). This definition also includes the T3 promoter 5′ AATTAACCCTCACTAAAG 3′ (see New England Biolabs, Ipswich, Mass.) or TATTTACCCTCACTAAAG (Adhya, et al., PNAS 78(1), 147-151 (1981). SP6 promoter has a sequence ATTTAGGTGACACTATAGAAGNG (Thermo Fisher Scientific, Waltham, Mass.). Other promoter sequences are known.

As used herein, the term “incubating”, refers to maintaining a reaction a suitable temperature and time to achieve the desired results, i.e., transcription. Reaction conditions suitable for the enzymes and reagents used in the present method are known (e.g. as described in the Examples herein) and, as such, suitable reaction conditions for the present method can be readily determined. These reactions conditions may change depending on the enzymes used (e.g., depending on their optimum temperatures, etc.).

As used herein, the term “composition” refers to a combination of reagents that may contain other reagents, e.g., glycerol, salt, dNTPs, etc., in addition to those listed. A composition may be in any form, e.g., aqueous or lyophilized, and may be at any state (e.g., frozen or in liquid form).

All publications, patents, and patent applications mentioned in this specification including PCT application number PCT/US2017/013179 filed Jan. 12, 2017; U.S. provisional application No. 62/278,161 filed Jan. 13, 2016; U.S. provisional application No. 62/416,770 filed Nov. 3, 2016; and U.S. application Ser. No. 15/594,090 filed May 12, 2017, are herein incorporated by reference to the same extent as if each individual publication, patent, or patent application was specifically and individually indicated to be incorporated by reference.

### DETAILED DESCRIPTION

Before various embodiments are described in greater detail, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Where a range of values is provided, it is understood that each intervening value, to a half of the unit of the lower limit unless the context clearly dictates otherwise, between the upper and lower limit of that range and any other stated or intervening value in that stated range is encompassed within the present disclosure.

Although any methods and materials similar or equivalent to those described herein can also be used in the practice or testing of the present teachings, the some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Variants and Compositions Containing the Same

Provided herein, in various embodiments, are isolated bacteriophage RNA polymerases belonging to the closely related family of bacteriophage RNA polymerases having at least 80% amino acid sequence identity with T7 RNA polymerase that may be engineered to contain one or more amino acid substitutions corresponding to those identified for T7 RNA polymerase described herein. The isolated bacteriophage RNA polymerase variants may be organized by their improved activity at temperatures of 42° C. and above compared to the corresponding wild type enzyme or wild type T7 RNA polymerase. In some embodiments, the variant: (i) may have an amino acid sequence is at least 80% sequence identity (e.g., at least 90%, at least 90%, at least 95%, at least 97%, at least 98% or at least 99% identity) to SEQ ID NO:1; and (ii) may comprise one or more (e.g., at least two, at least three, at least five, or at least ten) amino acid substitutions at one or more positions corresponding to positions 75, 83, 108, 109, 205, 206, 227, 281, 297, 312, 323, 327, 333, 340, 354, 362, 375, 388, 428, 446, 454, 461, 495, 510, 534, 567, 584, 591, 618, 642, 711, 724, 740, 788, 832, 834, 835, 843, 847, 849, 856, 863, 866, and 877 of SEQ ID NO:1 (wild-type T7 RNA polymerase), shown below:

SEQ ID NO: 1:
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLEAKHFKKNVEEQLNKRVGHVYKKAFMQVVEADMLSKGLLGGEA
WSSWHKEDSIHVGVRCIEMLIESTGMVSLHRQNAGVVGQDSETIELAPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDMNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEENHENIMACAKSPLENT
WWAEQDSPFCFLAFCFEYAGVQHHGLSYNCSLPLAFDGSCSGIQHFSAML
RDEVGGRAVNLLPSETVQDIYGIVAKKVNEILQADAINGTDNEVVTVTDE
NTGEISEKVKLGTKALAGQWLAYGVTRSVTKRSVMTLAYGSKEFGFRQQV
LEDTIQPAIDSGKGLMFTQPNQAAGYMAKLIWESVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLRDILESDFAFA

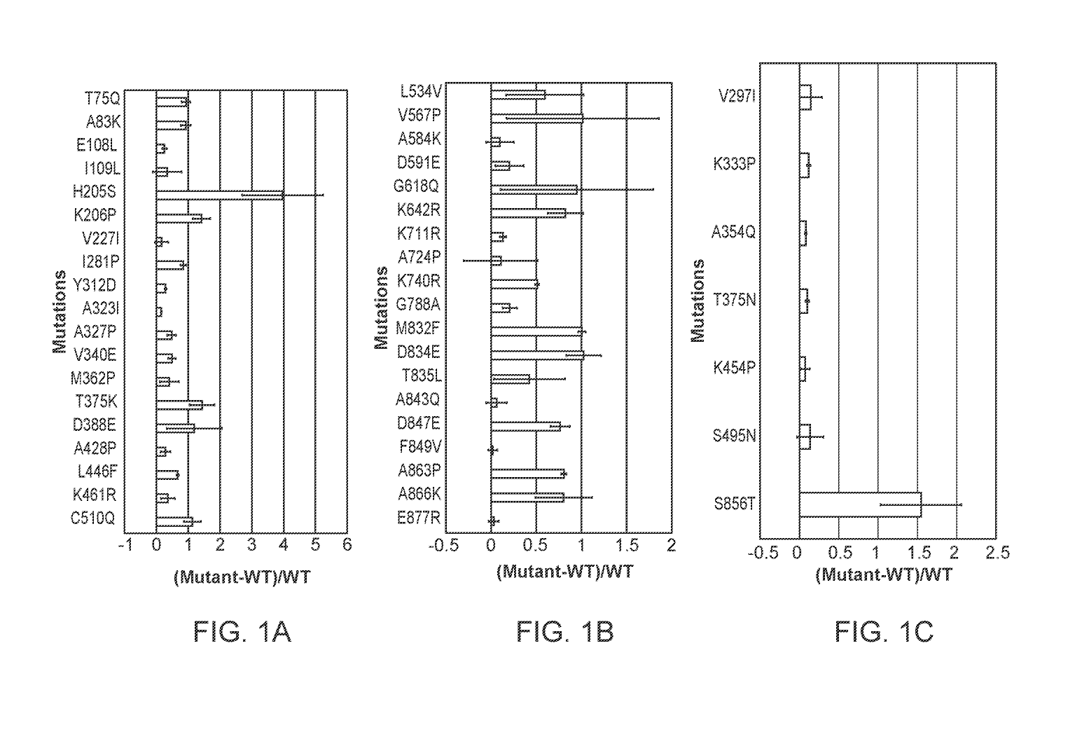

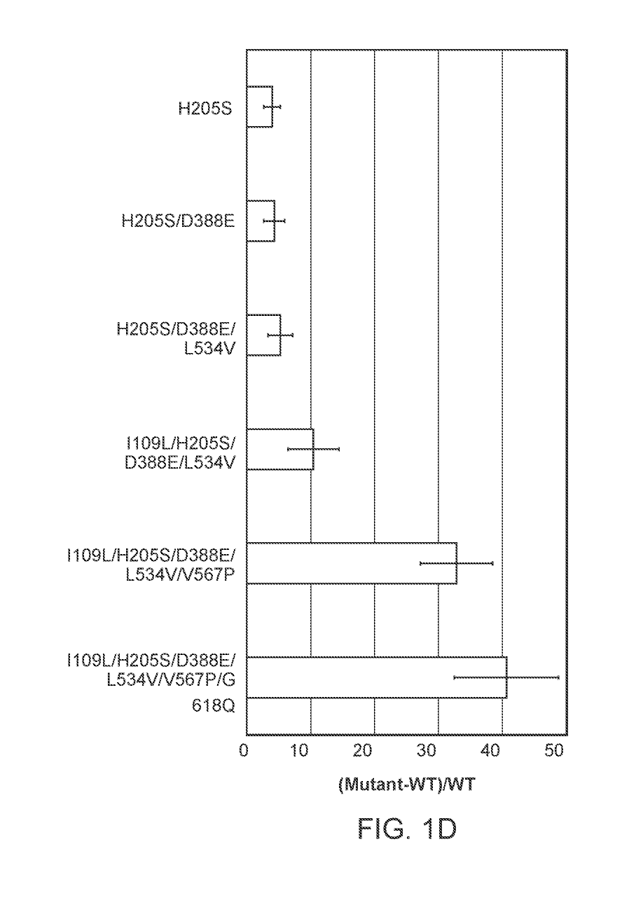

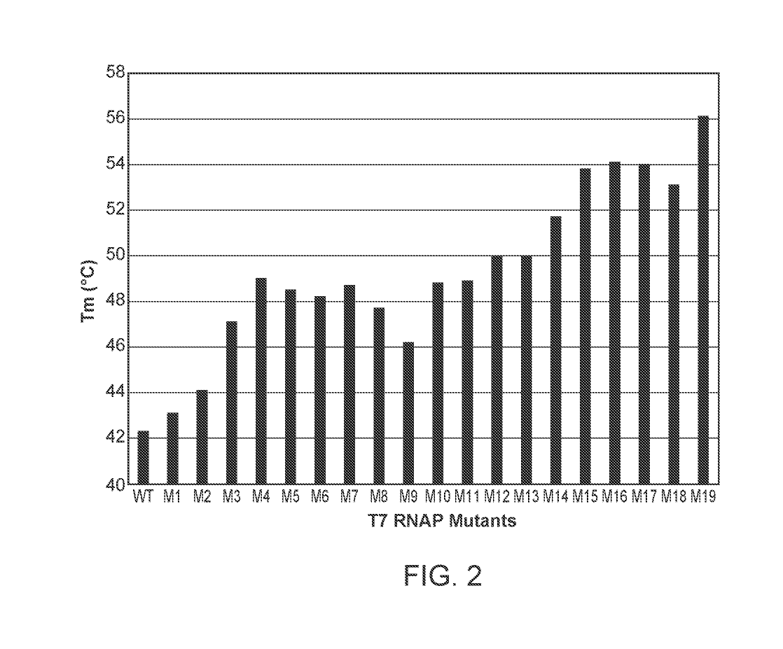

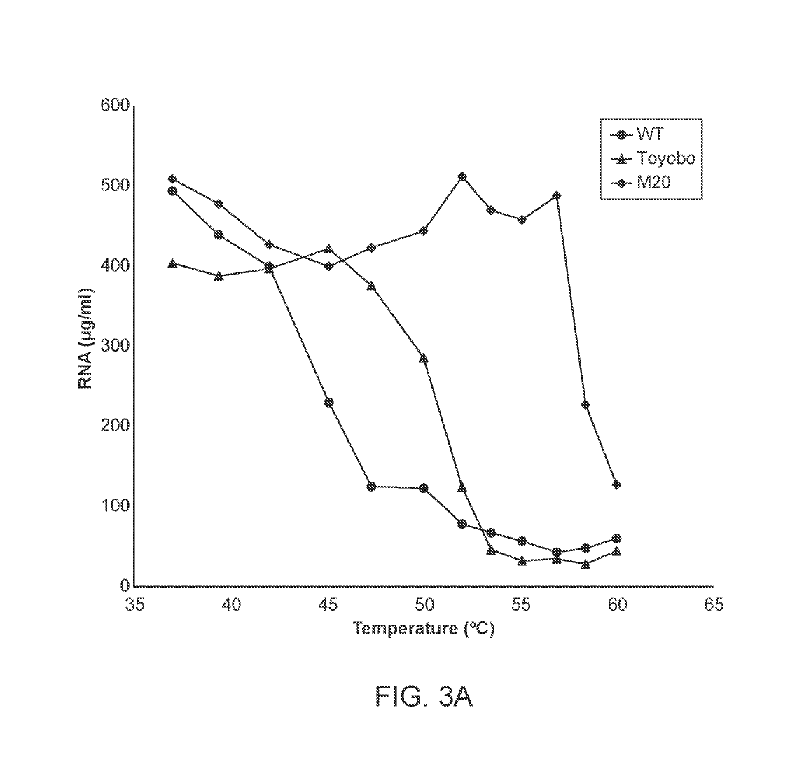

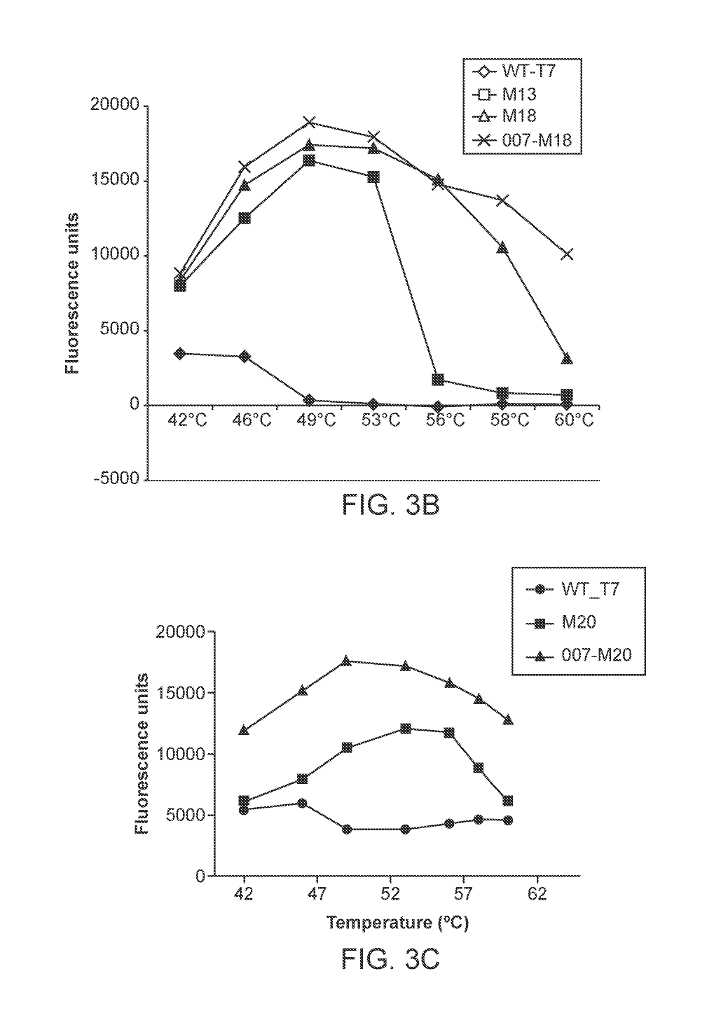

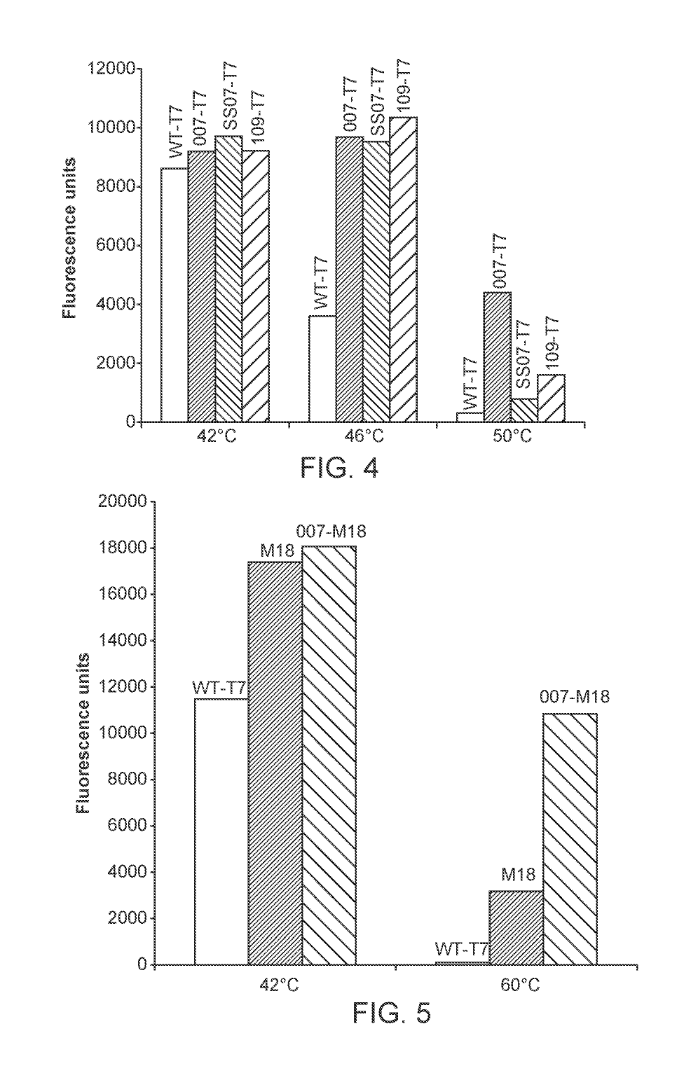

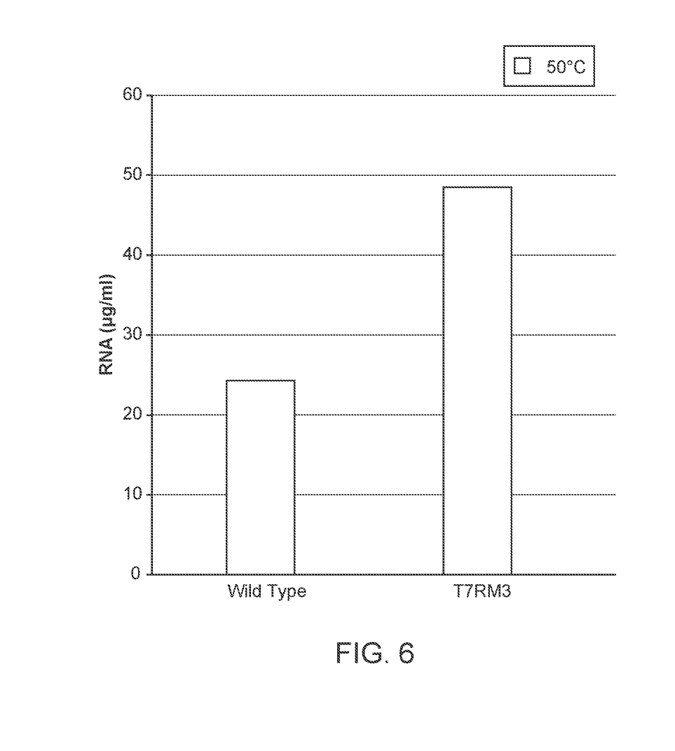

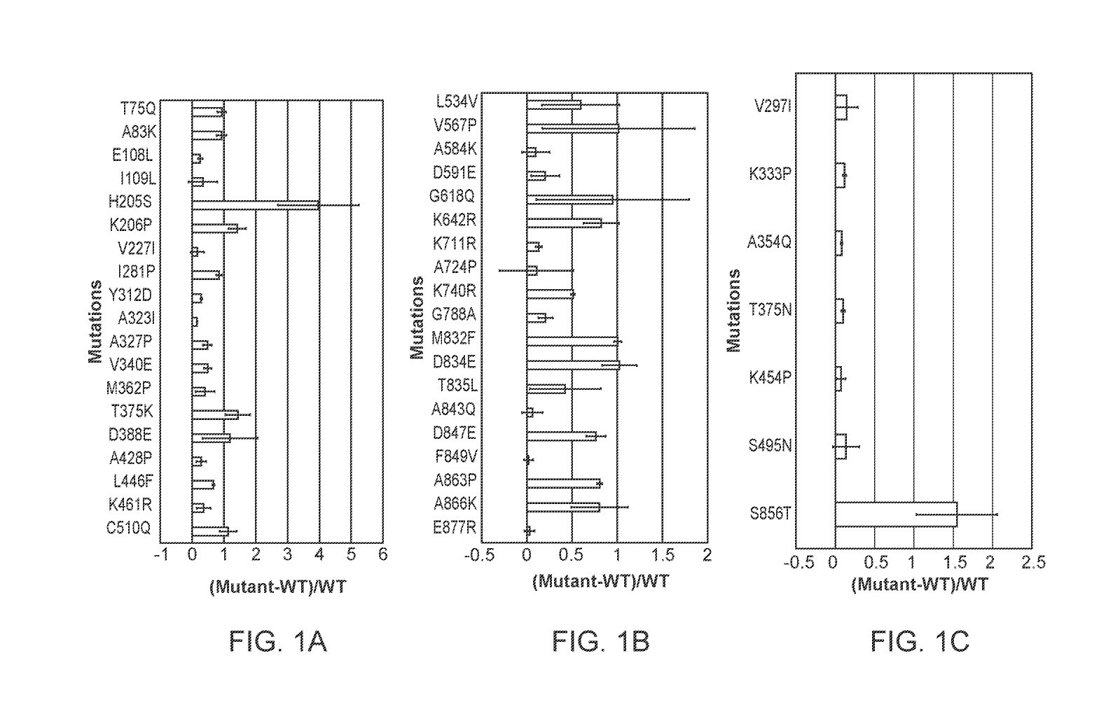

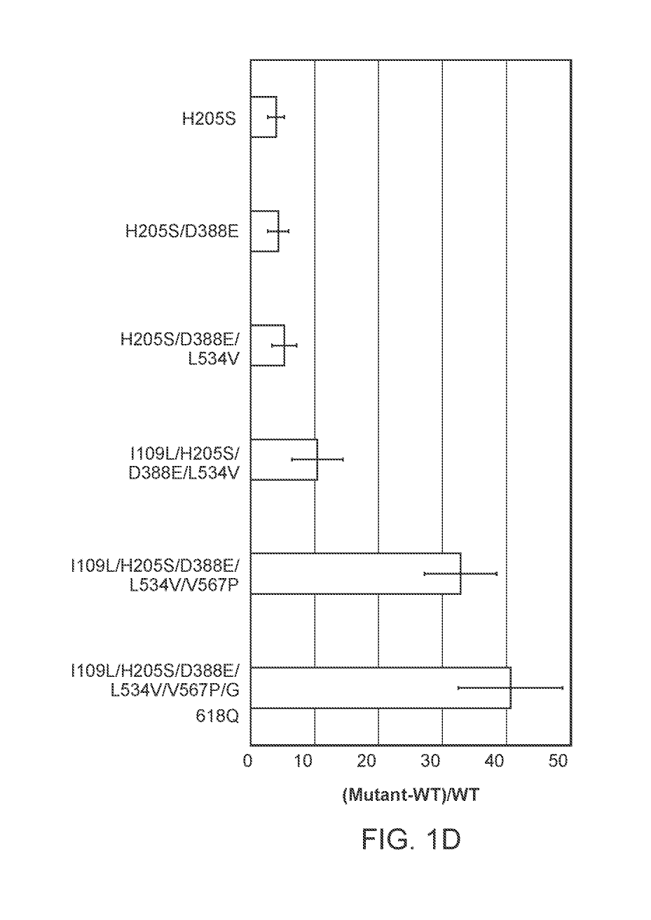

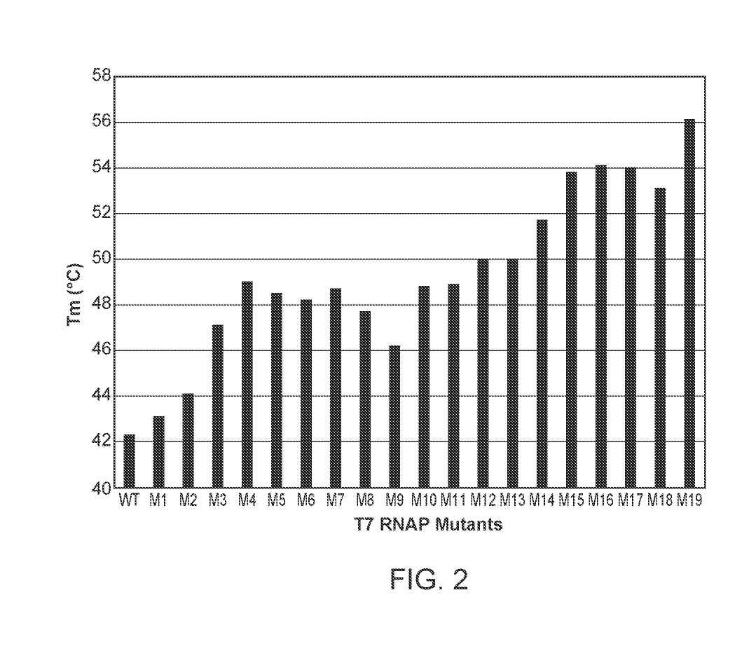

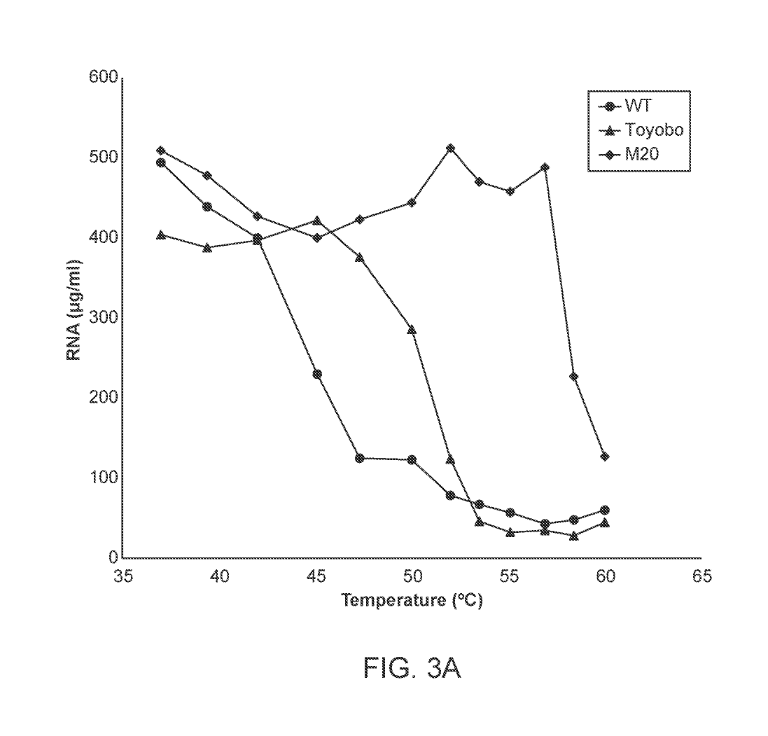

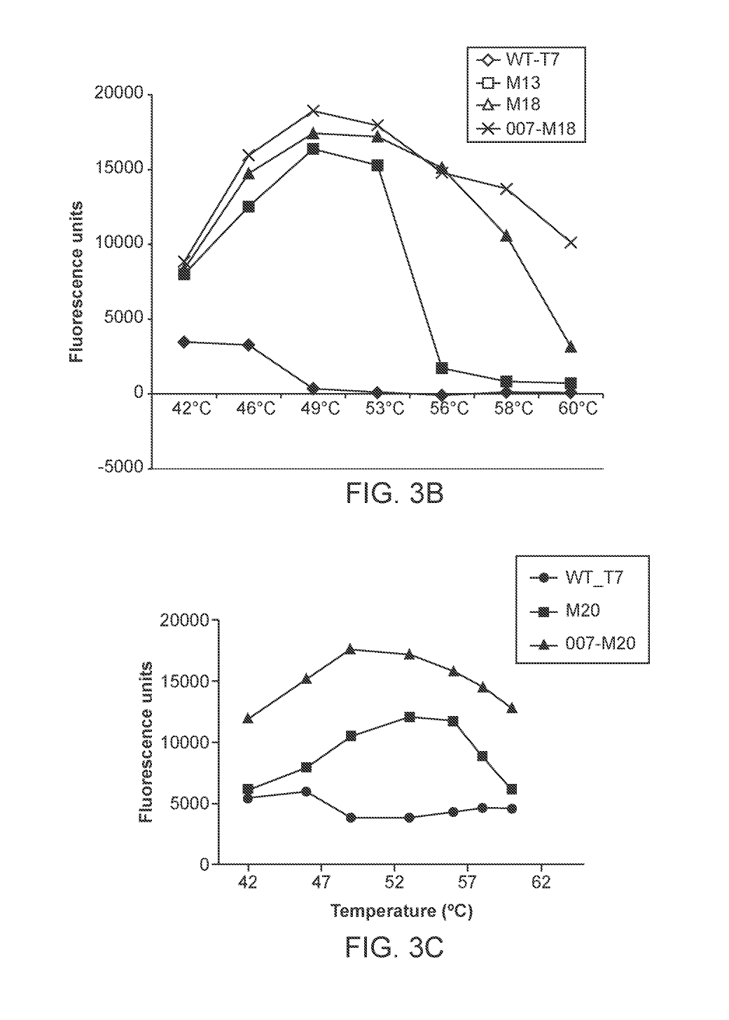

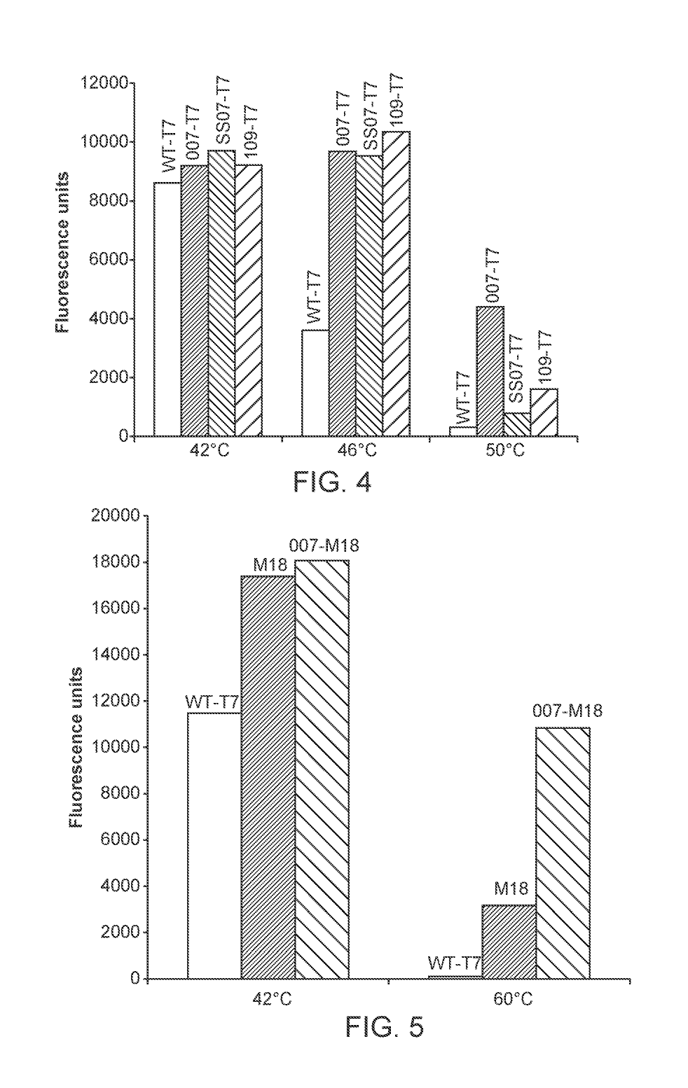

For example, in some embodiments, the variant may comprise substitutions at one or more (e.g., one, two, three, four, five or all six) positions corresponding to positions 109, 205, 388, 534, 567 and 618 of SEQ ID NO:1, as well as well as optionally one or more (e.g., at least two, at least three, at least five, or at least ten) other substitutions at other substitutions listed above. (see for example FIG. 1A-1D, FIG. 2, FIG. 3A-3C, FIG. 4 and FIG. 5).

In some embodiments, the isolated T7 RNA polymerase variant: (i) has an amino acid sequence is at least 80% sequence identity (e.g., at least 90%, at least 95%, at least 97%, at least 98% or at least 99% identity) to SEQ ID NO:1; and (ii) comprises one or more (e.g., at least two, at least three, at least five, or at least ten) of the following amino acid substitutions: T75Q, A83K, E108L, K206P, V227I, I281P, V297I, Y312D, A323I, A327P, K333P, V340E, A354Q, M362P, T375K, T375N, A428P, L446F, K454P, K461R, S495N, C510Q, A584K, D591E, K642R, K711R, A724P, K740R, G788A, M832F, D834E, T835L, A843Q, D847E, F849V, 5856T, A863P, A866K, and E877R, wherein the amino acid substitutions are at positions that correspond to positions in SEQ ID NO:1 (see for example FIG. 1A-1D, FIG. 2, FIG. 3A-3C, FIG. 4 and FIG. 5).

In some embodiments, the variant comprises one or more (e.g., one, two, three, four, five or all six) of the following amino acid substitutions: I109L, H205S, D388E, L534V, V567P and G618Q, wherein the amino acid substitutions are at positions that correspond to positions in SEQ ID NO:1, as well as well as optionally one or more (e.g., at least two, at least three, at least five, or at least ten) of the following amino acid substitutions: T75Q, A83K, E108L, K206P, V227I, I281P, V297I, Y312D, A323I, A327P, K333P, V340E, A354Q, M362P, T375K, T375N, A428P, L446F, K454P, K461R, S495N, C510Q, A584K, D591E, K642R, K711R, A724P, K740R, G788A, M832F, D834E, T835L, A843Q, D847E, F849V, S856T, A863P, A866K, and E877R, wherein the amino acid substitutions are at positions that correspond to positions in SEQ ID NO:1 (see for example FIG. 1A-1D, FIG. 2, FIG. 3A-3C, FIG. 4 and FIG. 5).

As would be apparent, RNA polymerase variants described herein have RNA polymerase activity and, as such, can catalyze the formation of RNA in the 5′→3′ direction using a DNA template. T7 RNA polymerase is a promoter-specific polymerase that transcribes downstream of a suitable promoter (e.g., TAATACGACTCACTATAG; SEQ ID NO:2). In certain embodiments, the non-natural bacteriophage RNA polymerase may activate transcription from a promoter that has at least 90% sequence identity with SEQ ID NO:2; AATTAACCCTCACTAAAG (SEQ ID NO:3); TATTTACCCTCACTAAAG (SEQ ID NO:4) or ATTTAGGTGACACTATAGAAGNG (SEQ ID NO:5). Transcription typically beginnings at the 3′ G nucleotide. The polymerase variants also preferably utilize Mg²⁺ ion as cofactor for the synthesis of RNA.

T7 RNA polymerase which is generally described in Maslak et al, Biochemistry 1994, 33: 6918-6924; Martin et al Prog. Nucleic Acid Res. Mol. Biol. 2005 80: 323-47; and Sousa et al Prog. Nucleic Acid Res. Mol. Biol. 2003 73: 1-41, is structurally related to other bacteriophage polymerases such as T3 polymerase (NP_523301.1) and SP6 polymerase (YP_004306655.1) as well as the RNA polymerases from Yersinia bacteriophage phiYeO3-12, Erwinia bacteriophage FE44, Kluyvera bacteriophage Kvp1, Enterobacteria bacteriophage K1F, Vibrio bacteriophage ICP3_2009_A and Pseudomonas bacteriophage PPPL-1. It is expected that the amino acid substitutions described herein may be transferred to other, related RNA polymerases and their variants with the same effect. As such, in certain embodiments, this disclosure provides a non-naturally occurring variant of a naturally occurring bacteriophage RNA polymerase, wherein the variant has an amino acid that is at least 80% identical to (e.g., at least 90%, at least 95% or at least 98% identical to) the naturally occurring bacteriophage RNA polymerase and comprises one or more amino acid substitutions relative the naturally occurring to bacteriophage RNA polymerase, wherein the one or more amino acid substitutions are at one or more position listed above.

In some embodiments, an isolated bacteriophage RNA polymerase variant with one or more amino acid substitutions has increased stability at 45° C. or greater relative to the wild type RNA polymerase. This is here exemplified by a T7 RNA polymerase variant, having one or more amino acid substitutions, and by a fusion between the bacteriophage RNA polymerase and a DNA binding protein such as described in Table 1 and in Example 3. In some embodiments, an isolated bacteriophage RNA polymerase variant exemplified by a T7 RNA polymerase variant may be used in an in vitro transcription reaction that is incubated at an elevated temperature (e.g., a temperature in the range of for example, 45° C. to 60° C., 45° C. to 50° C., 50° C. to 55° C. or 55° C. or 60° C.) to produce at least 10% more product, at least 20% more product, at least 50% more product, at least 100% more product, or at least 500% more product than an otherwise identical reaction containing the wild type RNA polymerase (e.g. SEQ ID NO:1 for T7 RNA polymerase) incubated under the same conditions.

Also provided is a composition, e.g., an aqueous composition comprising: i. an isolated bacteriophage RNA polymerase variant (e.g., a T7 RNA polymerase described herein) and ii. a buffering agent (e.g., Tris). In some embodiments, the composition may be a composition in which the polymerase can be stored. In these embodiments, the composition may optionally contain glycerol, salt (e.g., NaCl), EDTA, detergent (e.g., Triton X-100). In other embodiments, the composition may be a reaction mix. In these embodiments, the composition may further comprise ribonucleoside triphosphates (e.g., one, two, three or all four of ATP, UTP, GTP, CTP) and/or one or more modified nucleotides. In some embodiments, the composition may further comprise a template DNA molecule comprising: a bacteriophage promoter operably linked to a target nucleotide sequence to be transcribed. In some embodiments, the composition may comprise a population of such template DNA molecules, where each of the template molecules comprises a bacteriophage promoter upstream from a target nucleotide sequence to be transcribed. The bacteriophage promoter can be any of those described herein such as a T7 promoter, a T3 promoter or an SP6 promoter. A reaction mix composition may additionally contain 4-10 mM MgCl₂, e.g., 6 mM, MgCl₂.

In some embodiments, a variant may be fused to a DNA binding domain, the activity of the RNA polymerase may be enhanced at elevated temperatures by 50% or 100% or 150% or 200% or more (see for example, FIG. 4 or FIG. 5).

Kits

Also provided is a kit comprising: i. an isolated bacteriophage RNA polymerase variant as described herein; and ii. a reaction buffer. In some embodiments, the kit may further comprise one or more ribonucleoside triphosphates (e.g., one, two, three or all four of ATP, UTP, GTP, CTP). The components of the kit may be combined in one container, or each component may be in its own container. For example, the components of the kit may be combined in a single reaction tube or in one or more different reaction tubes. Further details of the components of this kit are described above. The kit may also contain other reagents described above and below that may be employed in the method depending on how the method is going to be implemented. In some embodiments, the kit may comprise of a variant as described above and a buffer in which the variant is active, or a concentrated form thereof.

In addition to above-mentioned components, the subject kit may further include instructions for using the components of the kit to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Methods

Also provided is a method for synthesizing an RNA molecule. In some embodiments, this method may comprise (a) combining an isolated bacteriophage RNA polymerase variant described herein with ribonucleoside triphosphates and/or modified nucleotides and a template DNA molecule comprising a promoter operably linked to a target nucleotide sequence to be transcribed, to produce a reaction mix; and (b) incubating the reaction mix to transcribe the template DNA molecule into RNA. In some embodiments, the incubating may be done at a temperature of at least 45° C. (e.g., in the range of 45° C. to 60° C., 45° C. to 50° C., 50° C. to 55° C. or 55° C. or 60° C.) to transcribe the DNA into RNA. The DNA can be single- or double-stranded and should have a promoter recognized by the polymerase. In one embodiment, the method includes a T7 RNA polymerase variant and a T7 promoter or T3 promoter or variants thereof.

In some embodiments, RNA polymerase may be used in for NASBA (Nucleic Acid Sequence Based Amplification) to amplify RNA sequences. NASBA was initially described by Compton (Nature, 350 (6313):91-92 (1991)) and has been used as a rapid diagnostic tests for several pathogenic viruses with RNA genomes, e.g. influenza A, foot-and-mouth disease virus, severe acute respiratory syndrome (SARS)-associated coronavirus, human bocavirus (HBoV) and also parasites like Trypanosoma brucei as well as other viruses such as HIV-1 (see, e.g., Kievits Journal of Virological Methods. 1991 35: 273-86). NASBA can be used for medical diagnostics, where it has been shown to give quicker results than PCR, and it can also be more sensitive. NASBA's is an isothermal reaction that is typically run at a constant temperature of at least 41° C. When a present variant is used, the incubation temperature can be increased to above at least 45° C. (e.g., in the range of 45° C. to 60° C., 45° C. to 50° C., 50° C. to 55° C. or 55° C. or 60° C.). In some implementations, when the RNA template is added the reaction mixture, a primer containing a promoter sequence hybridizes to a complementary site at the 3′ end of the template, and reverse transcriptase synthesizes the opposite, complementary DNA strand. RNAse H destroys the RNA template from the DNA-RNA compound, and a second primer attaches to the 5′ end of the DNA strand. Reverse transcriptase again synthesizes another DNA strand from the attached primer resulting in double stranded DNA. A T7 RNA polymerase variant can continuously produce complementary RNA strands of this template, which results in amplification. The amplicons, however, are antisense to the original RNA template. A higher incubation temperature results in less non-specific binding of DNA primers to the RNA. In some embodiments, the reaction may contain temperature-sensitive inhibitor of the polymerase, thereby allowing the polymerase to remain inactive until the temperature is raised.

Examples of closely related bacteriophage RNA polymerases are provided below. Mutations identified for T7 RNA polymerase that improve thermostability and/or activity are expected to have a corresponding effect when positioned in closely related bacteriophage RNA polymerases in corresponding positions.

Enterobacteria bacteriophage 13a
(SEQ ID NO: 52)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSVDNTTVQAVASAIGRAIEDEAR
FGRIRDLEAKHFKKNVEEQLNKRVGHVYKKAFMQVVEADMLSKGLLGGEA
WSSWHKEDSIHVGVRCIEMLIESTGMVSLHRQNAGVVGQDSETIELAPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDTNPDALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEDNHENIMACAKSPLENT
WWAEQDSPFCFLAFCFEYAGVQHHGLSYNCSLPLAFDGSCSGIQHFSAML
LDEIGGRAVNLLPSETVQDIYGIVAKKVNVILQADVINGTDNEVVTVTDE
NTGEISEKVKLGTKALAGQWLAYGVTRSVTKRSVMTLAYGSKEFGFRQQV
LEDTIQPAIDSGKGLMFTQPNQAAGYMAKLIWEAVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLQDILKSDFAFA
Yersinia bacteriophage YpP-R
(SEQ ID NO: 53)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLEAKHFKKNVEEQLNKRVGHVYKKAFMQVVEADMLSKGLLGGEA
WSSWHKEDSIHVGVRCIEMLIESTGMVNLHRQNAGVVGQDSETIELTPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDTNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEDNHENIMACAKSPLENT
WWAEQDSPFCFLAFCFEYAGVQHHGLSYNCSLPLAFDGSCSGIQHFSAML
LDEVGGLAVNLLPSATVQDIYGIVAKKVNVILQADVINGTDNEVVTVTDE
NTGEISEKVKLGTKALAGQWLAYGVTRSVTKRSVMTLAYGSKEFGFRQQV
LEDTIQPAIDSGKGLMFTQPNQAAGYMAKLIWEAVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLQDILKSDFAFA
Yersinia bacteriophage R
(SEQ ID NO: 54)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLEAKHFKKNVEEQLNKRVGHVYKKAFMQVVEADMLSKGLLGGEA
WSSWHKEDSIHVGVRCIEMLIESTGMVNLHRQNAGVVGQDSETIELTPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDTNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEDNHENIMACAKSPLENT
WWAEQDSPFCFLAFCFEYAGVQHHGLSYNCSLPLAFDGSCSGIQHFSAML
LDEVGGLAVNLLPSATVQDIYGIVAKKVNVILQADVINGTDNEVVTVTDE
NTGEIPEKVKLGTKALAGQWLAYGVTRSVTKRSVMTLAYGSKEFGFRQQV
LEDTIQPAIDSGKGLMFTQPNQAAGYMAKLIWEAVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLQDILKSDFAFA
Yersinia bacteriophage phiA1122
(SEQ ID NO: 55)
MNTINIAKNDFSDIELAAIPFNTLADHYGERLAREQLALEHESYEMGEAR
FRKMFERQLKAGEVADNAAAKPLITTLLPKMIARINDWFEEVKAKRGKRP
TAFQFLQEIKPEAVAYITIKTTLACLTSADNTTVQAVASAIGRAIEDEAR
FGRIRDLEAKHFKKNVEEQLNKRVGHVYKKAFMQVVEADMLSKGLLGGEA
WSSWHKEDSIHVGVRCIEMLIESTGMVNLHRQNAGVVGQDSETIELTPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDTNPEALTAWKRAAAAVYRKDKARKSRRISLEF
MLEQANKFANHKAIWFPYNMDWRGRVYAVSMFNPQGNDMTKGLLTLAKGK
PIGKEGYYWLKIHGANCAGVDKVPFPERIKFIEDNHENIMACAKSPLENT
WWAEQDSPFCFLAFCFEYAGVQHHGLSYNCSLPLAFDGSCSGIQHFSAML
LDEVGGLAVNLLPSATVQDIYGIVAKKVNVILQADVINGTDNEVVTVTDE
NTGEIPEKVKLGTKALAGQWLAYGVTRSVTKRSVMTLAYGSKEFGFRQQV
LEDTIQPAIDSGKGLMFTQPNQAAGYMAKLIWEAVSVTVVAAVEAMNWLK
SAAKLLAAEVKDKKTGEILRKRCAVHWVTPDGFPVWQEYKKPIQTRLNLM
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLQDILKSDFAFA
Escherichia bacteriophage CICC 80001
(SEQ ID NO: 56)
MNTINIAKNDFSDIELAAIPFNTLADHYGERLAREQLALEHESYEMGEAR
FRKMFERQLKAGEVADNAAAKPLITTLLPKMIARINDWFEVVKAKRGKRP
TAFQFLQEIKPEAVAYITIKTTLACLTSADNTTVQAVASAIGRAIEDESR
FGRIRDLEAKHFKKNVEEQLNKRVGHVYRKAFMQVVEADMLSKGLMGGEA
WSSWHKEDSIHVGVRCIEMLIESTGMVSLHRQNAGVVGQDSETIELAPEY
AEAIATRAGALAGISPMFQPCVVPPKPWTGITGGGYWANGRRPLALVRTH
SKKALMRYEDVYMPEVYKAINIAQNTAWKINKKVLAVANVITKWKHCPVE
DIPAIEREELPMKPEDIDTNPEALTAWKRAAAAVYRKDKARKSRRISLEF
MLEQANKFANHKSIWFPYNMDWRGRVYAVSMFNPQGNDMTKGLLTLAKGK
PIGKEGYYWLKIHGANCAGVDKVPFPERIKFIEDNHENIMACAKSPLENT
WWAEQDSPFCFLAFCFEYAGVQHHGLSYNCSLPLAFDGSCSGIQHFSAML
RDEVGGLAVNLLPSETVQDIYGIVAKKVNVILQEDVINGTDNEVVTVTDE
NTGEISEKVKLGTKALAGQWLAYGVTRSVTKRSVMTLAYGSKEFGFRQQV
LEDTIQPAIDSGKGLMFTQPNQAAGYMAKLIWEAVSVTVVAAVEAMNWLK
SAAKLLAAEVKDKKTGEILRKRCAVHWVTPDGFPVWQEYKKPIQTRLNLI
FLGQFRLQPTINTNKDSEIDAHKQESGIAPNFVHSQDGSHLRKTVVWAHE
KYGIESFALIHDSFGTIPADAANLFKAVRETMVDTYESCDVLADFYDQFA
DQLHESQLDKMPALPAKGNLNLQDILKSDFAFA
Yersinia bacteriophage YpsP-G
(SEQ ID NO: 57)
MTERTDGLKKGYMPNGTLYAANRRLVRTWRENNLELAAIPFNTLADHYGE
RLAREQLALEHESYEMGEARFRKMFERQLKAGEVADNAAAKPLITTLLPK
MIARINDWFEEVKAKRGKRPTAFQFLQEIKPEAVAYITIKTTLACLTSAD
NTTVQAVASAIGRAIEDEARFGRIRDLEAKHFKKNVEEQLNKRVGHVYKK
AFMQVVEADMLSKGLLGGEAWSSWHKEDSIHVGVRCIEMLIESTGMVNLH
RQNAGVVGQDSETIELTPEYAEAIATRAGALAGISPMFQPCVVPPKPWTG
ITGGGYWANGRRPLALVRTHSKKALMRYEDVYMPEVYKAINIAQNTAWKI
NKKVLAVANVITKWKHCPVEDIPAIEREELPMKPEDIDTNPEALTAWKRA
AAAVYRKDKARKSRRISLEFMLEQANKFANHKAIWFPYNMDWRGRVYAVS
MFNPQGNDMTKGLLTLAKGKPIGKEGYYWLKIHGANCAGVDKVPFPERIK
FIEDNHENIMACAKSPLENTWWAEQDSPFCFLAFCFEYAGVQHHGLSYNC
SLPLAFDGSCSGIQHFSAMLLDEVGGLAVNLLPSATVQDIYGIVAKKVNV
ILQADVINGTDNEVVTVTDENTGEISEKVKLGTKALAGQWLAYGVTRSVT
KRSVMTLAYGSKEFGFRQQVLEDTIQPVIDSGKGLMFTQPNQAAGYMAKL
IWEAVSVTVVAAVEAMNWLKSAAKLLAAEVKDKKTGEILRKRCAVHWVTP
DGFPVWQEYKKPIQTRLNLMFLGQFRLQPTINTNKDSEIDAHKQESGIAP
NFVHSQDGSHLRKTVVWAHEKYGIESFALIHDSFGTIPADAANLFKAVRE
TMVDTYESCDVLADFYDQFADQLHESQLDKMPALPAKGNLNLQDILKSDF
AFA
Salmonella bacteriophage Vi06
(SEQ ID NO: 58)
MNTISITKNDFSDIELAAIPFNTLADHYGERLAREQLALEHESYEMGEVR
FRKMFERQLKAGEIADNDATKPLITTLLPKMIARINSWFKEVQAKCGKRP
TAFQFLQGIKPEAIAYITIKTTLARLTSMDNTTVQAVASAIGRAIEDEAR
FGRIRDLEAKHFKKNVEEQLNKRVGHVYKKAFMQVIEADMLSKGLLGGES
WSSWHKEDSIHVGVRCIEMLIESTGMVSLHRQNAGVVGQDSETIELAPEY
AEAIATRAGALAGISPMFQPCVVPPKPWTSISGGGYWANGRRPLALVRTH
SKKALMRYADVYMPEVYKAVNIAQNTAWRINKKVLAVANVVTKWKHCPVD
YIPTIEREELPMKPEDIDTNPEALASWKRAAAAVYRKDKARKSRRMSLEF
MLEQANKFANHRAIWFPYNMDWRGRVYAVSMFNPQGNDMTKGLLTLAKGK
PIGKEGFYWLKIHGANCAGVDKVPFPERIKFIEDNHENILACAKSPLENT
WWSEQDSPFCFLAFCFEYAGGQHHGLSYNCSLPLAFDGSCFGIQHFSVML
RDEVGGRAVNLLPSETVQDIYGIVAKKVNEILQVDMINGTDNEVVTVTDD
KTGEIYEKIKLGTKELAGQWLAYGVTRSVTKRSVMTLAYGSKEFGFRQQV
LEDTIQPAIDSGKGLMFTHPNQAAGYMAKLIWEAVSVTVVAAVEAMNWLK
SAAKLLAVEVKDRKTGEILRKRCAVHWTTPDGFPVWQEYKKPVQTRLNLI
FLGQFRLQPTINTNRDSEIDAYKQESGIAPNFVHSQDGSHLRKTVVWAHE
KYGIDSFALIHDSFGTIPADAANLFKAVRETMVATYESCDVLADFYAQFA
DQLHKSQLDKMPVLPSKGNLNLQDILKSDFAFA
Stenotrophomonas bacteriophage IME15
(SEQ ID NO: 59)
MTVIAIEKNDFSDVELAVIPFNTLADHYGEKLAREQLALEHEAYEMGEAR
FRKIFERQLKAGEVADNAAAKPLVATLLPKMIERIHAWFEEVSAKRGKRP
TAFKFLQEVKPEAIAYITIKTVLGTLTSAEQTTVQAAASAVGRAIEDEAR
FGRIRDLEAKHFKKNVEEQLNKRVGHVYKKAFLQVVEADMLSKGLMGGEA
WSSWHKEDSIHVGVRCIEMLIEATGLVVLERQNAGVVGADAETLSLASEY
ADAIATRAGALAGISPMYQPCVVPPKPWTTVTGGGWVANGRRPLALVRTH
GKKALMRYEDVYMPEVYKAVNLAQSTAWKINKKVLAVANEITKWKHCPVE
DIPAIEREELPVKPDDIDENPEALTNWKRAAAAVYRKDKARKSRRLSLEF
MLEQANKFANHKAIWFPYNMDWRGRVYAVSMFNPQGNDMTKGLLTLAKGK
AIGKEGFYWLKIHGANCAGVDKVPFPERIKFIEDNHEHIMASAKNPLEYT
WWAEQDSPFCFLAFCFEYAGVMHHGLSYNCSLPLAFDGSCSGIQHFSAML
RDEVGGRAVNLLPSETVQDIYGIVAKKVNEIMQRDVISGTDDELVTETDK
TTGEITEKAVLGTRTLAGQWLAYGANRSVTKRSVMTLAYGSKEFGFRQQV
LEDTIRPAIDSGKGLMFTIPNQAAGYMAKLIWDSVSVTVVAAVEAMKWLQ
SAAKLLAAEVKDKKTGEVLRNRCAVHWVTPDGFPVWQEYRKPLQTRLNLM
FLGQFRLQPTINTNKDSGIDAHKQESGIAPNFVHSQDGSHLRKTVVWAHE
KYGIESFALIHDSFGTIPADAGNLFKAVRETMVDTYENCDVLADFYEQFA
DQLHESQLDKMPALPKKGNLNLRDILESDFAFA
Citrobacter bacteriophage 5H2
(SEQ ID NO: 60)
MNIIENIEKNDFSEIELAAIPFNTLADHYGSALAREQLALEHESYELGER
RFLKMLERQAKAGEIADNAAAKPLLATLLPKLTARIVEWLEEYASKKGRK
PVAYAPLQLLKPEASAFITLKVILASLTSTNMTTIQAAAGMLGKAIEDEA
RFGRIRDLEAKHFKKHVEEQLNKRHGQVYKKAFMQVVEADMIGRGLLGGE
AWSSWDKETTMHVGIRLIEMLIESTGLVELQRHNAGNAGSDHEALQLAQE
YVDVLAKRAGALAGISPMFQPCVVPPKPWVSITGGGYWANGRRPLALVRT
HSKKGLMRYEDVYMPEVYKAVNIAQNTAWKINKKVLAVVNEIVNWKNCPV
ADIPSLERQELPPKPDDIDTNEAALKEWKKAAAGVYRLDKARVSRRISLE
FMLEQANKFANKKAIWFPYNMDWRGRVYAVPMFNPQGNDMTKGLLTLAKG
KPIGEEGFYWLKIHGANCAGVDKVPFPERIAFIEKHVDDILACAKDPINN
TWWAEQDSPFCFLAFCFEYAGVAHHGLSYNCSLPLAFDGSCSGIQHFSAM
LRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQ
VLDDTIQPAIDSGKGLMFTQPNQAAGYMAKLIWDAVSVTVVAAVEAMNWL
KSAAKLLAAEVKDKKTKEILRHRCAVHWTTPDGFPVWQEYRKPLQKRLDM
IFLGQFRLQPTINTLKDSGIDAHKQESGIAPNFVHSQDGSHLRMTVVYAH
EKYGIESFALIHDSFGTIPADAGKLFKAVRETMVITYENNDVLADFYDQF
ADQLHETQLDKMPPLPKKGNLNLQDILKSDFAFA
Enterobacter bacteriophage E-4
(SEQ ID NO: 61)
MNIIENIEKNDFSEIELAAIPFNTLADHYGSALAREQLALEHESYELGER
RFLKMLERQAKAGEIADNAAAKPLLATLLPKLTARIVEWLEEYASKKGRK
PSAYAPLQLLKPEASAFITLKVILASLTSTNMTTIQAAAGMLGKAIEDEA
RFGRIRDLEAKHFKKHVEEQLNKRHGQVYKKAFMQVVEADMIGRGLLGGE
AWSSWDKETTMHVGIRLIEMLIESTGLVELQRHNAGNAGSDHEALQLAQE
YVDVLAKRAGALAGISPMFQPCVVPPKPWVSITGGGYWANGRRPLALVRT
HSKKGLMRYEDVYMPEVYKAVNIAQNTAWKINKKVLAVVNEIVNWKNCPV
ADIPSLERQELPPKPDDIDTNEAALKEWKKAAAGIYRLDKARVSRRISLE
FMLEQANKFANKKAIWFPYNMDWRGRVYAVPMFNPQGNDMTKGLLTLAKG
KPIGEEGFYWLKIHGANCAGVDKVPFPERIAFIEKHVDDILACAKDPINN
TWWAEQDSPFCFLAFCFEYAGVAHHGLSYNCSLPLAFDGSCSGIQHFSAM
LRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQ
VLDDTIQPAIDSGKGLMFTQPNQAAGYMAKLIWDAVSVTVVAAVEAMNWL
KSAAKLLAAEVKDKKTKEILRHRCAVHWTTPDGFPVWQEYRKPLQKRLDM
IFLGQFRLQPTINTLKDSGIDAHKQESGIAPNFVHSQDGSHLRMTVVYAH
EKYGIESFALIHDSFGTIPADAGKLFKAVRETMVLTYENNDVLADFYDQF
ADQLHETQLDKMPPLPKKGNLNLQDILKSDFAFA
Yersinia bacteriophage phiYe-F10
(SEQ ID NO: 62)
MNIIENIEKNDFSEIELAAIPFNTLADHYGSALAREQLALEHESYELGER
RFLKMLERQAKAGEIADNAAAKPLLATLLPKLTARIVEWLEEYASKKGRK
PVAYAPLQSLKPEASAFITLKVILASLTSTNMTTIQAAAGMLGKAIEDEA
RFGRIRDLEAKHFKKHVEEQLNKRHGQVYKKAFMQVVEADMIGRGLLGGE
AWSSWDKETTMHVGIRLIEMLIESTGLVELQRHNAGNAGSDHEALQLAQE
YVDVLAKRAGALAGISPMFQPCVVPPKPWVSITGGGYWANGRRPLALIRT
HSKKGLMRYEDVYMPEVYKAVNIAQNTAWKINKKVLAVVNEIVNWKNCPV
ADIPSLERQELPPKPDDIDTNEAALKEWKKAAAGVYRLDKARVSRRISLE
FMLEQANKFASKKAIWFPYNMDWRGRVYAVPMFNPQGNDMTKGLLTLAKG
KPIGEEGFYWLKIHGANCAGVDKVPFPERIAFIEKHVDDILACAKDPINN
TWWAEQDSPFCFLAFCFEYAGVAHHGLSYNCSLPLAFDGSCSGIQHFSAM
LRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQ
VLDDTIQPAIDSGKGLMFTQPNQAAGYMAKLIWDAVSVTVVAAVEAMNWL
KSAAKLLAAEVKDKKTKEILRHRCAVHWTTPDGFPVWQEYRKPLQKRLDM
IFLGQFRLQPTINTLKDSGIDAHKQESGIAPNFVHSQDGSHLRMTVVYAH
EKYGIESFALIHDSFGTIPADAGKLFKAVRETMVITYENNDVLADFYDQF
ADQLHETQLDKMPPLPKKGNLNLQDILKSDFAFA
Citrobacter bacteriophage phiCFP-1
(SEQ ID NO: 63)
MNIIENIEKNDFSEIELAAIPFNTLADHYGSALAREQLALEHESYELGER
RFLKMLERQAKAGEIADNAAAKPLLATLLPKLTARIVEWLEEYDSKKGRK
PVAYAPLQLLKPEASAFITLKVILASLTSTNMTTIQAAAGMLGKAIEDEA
RFGRIRDLEAKHFKKHVEEQLNKRHGQVYKKAFMQVVEADMIGRGLLGGE
AWSSWDKETTMHVGIRLIEMLIESTGLVELQRHNAGNAGSDHEALQLAQE
YVDVLAKRAGALAGISPMFQPCVVPPKPWVAITGGGYWANGRRPLALVRT
HSKKGLMRYEDVYMPEVYKAVNIAQNTAWKINKKVLAVVNEIVNWKNCPV
ADIPSLERQELPPKPDDIDTNEAALKEWKKAAAGIYRLDKARVSRRISLE
FMLEQANKFASKKAIWFPYNMDWRGRVYAVPMFNPQGNDMTKGLLTLAKG
KPIGEEGFYWLKIHGANCAGVDKVPFPERIAFIEKHVDDILACAKDPINN
TWWAEQDSPFCFLAFCFEYAGVAHHGLSYNCSLPLAFDGSCSGIQHFSAM
LRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQ
VLDDTIQPAIDSGKGLMFTQPNQAAGYMAKLIWDAVSVTVVAAVEAMNWL
KSAAKLLAAEVKDKKTKEILRHRCAVHWTTPDGFPVWQEYRKPLQKRLDM
IFLGQFRLQPTINTLKDSGIDAHKQESGIAPNFVHSQDGSHLRMTVVYAH
EKYGIESFALIHDSFGTIPADAGKLFKAVRETMVITYENNDVLADFYDQF
ADQLHETQLDKMPPLPKKGNLNLQDILKSDFAFA
Citrobacter bacteriophage SH1
(SEQ ID NO: 64)
MNIIENIEKNDFSEIELAAIPFNTLADHYGSALAREQLALEHESYELGER
RFLKMLERQAKAGEIADNAAAKPLLATLLPKLTARIVEWLEEYASKKGRK
PVAYAPLQLLKPEASAFITLKVILASLTSTNMTTIQAAAGMLGKAIEDEA
RFGRIRDLEAKHFKKHVEEQLNKRHGQVYKKAFMQVVEADMIGRGLLGGE
AWSSWDKETTMHVGIRLIEMLIESTGLVELQRHNAGNAGSDHEALQLAQE
YVDVLAKRAGALAGISPMFQPCVVPPKPWVSITGGGYWANGRRPLALVRT
HSKKGLMRYEDVYMPEVYKAVNIAQNTAWKINKKVLAVVNEIVNWKNCPV
ADIPSLERQELPPKPDDIDTNEAALKEWKKAAAGIYRLDKARVSRRISLE
FMLEQANKFASKKAIWFPYNMDWRGRVYAVPMFNPQGNDMTKGLLTLAKG
KPIGEEGFYWLKIHGANCAGVDKVPFPERIAFIEKHVDDILACAKDPINN
TWWAEQDSPFCFLAFCFEYAGVAHHGLSYNCSLPLAFDGSCSGIQHFSAM
LRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQ
VLDDTIQPAIDSGKGLMFTQPNQAAGYMAKLIWDAVSVTVVAAVEAMNWL
KSAAKLLADEVKDKKTKEILRHRCAVHWTTPDGFPVWQEYRKPLQKRLDM
IFLGQFRLQPTINTLKDSGIDAHKQESGIAPNFVHSQDGSHLRMTVVYAH
EKYGIESFALIHDSFGTIPADAGKLFKAVRETMVITYENNDVLADFYDQF
ADQLHETQLDKMPPLPKKGNLNLQDILKSDFAFA
Salmonella bacteriophage phiSG-JL2
(SEQ ID NO: 65)
MNIIENIEKNDFSEIELAAIPFNTLADHYGSALAREQLALEHESYELGER
RFLKMLERQAKAGEIADNAAAKPLLATLLPKLTARIVEWLEEYASKKGRK
PVAYAPLQLLKPEASAFITLKVILASLTSTNMTTIQAAAGMLGKAIEDEA
RFGRIRDLEAKHFKKHVEEQLNKRHGQVYKKAFMQVVEADMIGRGLLGGE
AWSSWDKETTMHVGIRLIEMLIESTGLVELQRHNAGNAGSDHEALQLAQE
YVDVLAKRAGALAGISPMFQPCVVPPKPWVAITGGGYWANGRRPLALVRT
HSKKGLMRYEDVYMPEVYKAVNIAQNTAWKINKKVLAVVNEIVNWKNCPV
ADIPSLERQELPPKPDDIDTNEAALKEWKKAAAGVYRLDKARVSRRISLE
FMLEQANKFASKKAIWFPYNMDWRGRVYAVPMFNPQGNDMTKGLLTLAKG
KPIGEEGFYWLKIHGANCAGVDKVPFPERIAFIEKHVDDILACAKDPINN
TWWAEQDSPFCFLAFCFEYAGVAHHGLSYNCSLPLAFDGSCSGIQHFSAM
LRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQ
VLDDTIQPAIDSGKGLMFTQPNQAAGYMAKLIWDAVSVTVVAAVEAMNWL
KSAAKLLAAEVKDKKTKEILRHRCAVHWTTPDGFPVWQEYRKPLQKRLDM
IFLGQFRLQPTINTLKDSGIDAHKQESGIAPNFVHSQDGSHLRMTVVYAH
EKYGIESFALIHDSFGTIPADAGKLFKAVRETMVLTYENNDVLADFYDQF
ADQLHETQLDKMPPLPKKGKLNLQDILKSDFAFA
Yersinia bacteriophage phiYe03-12
(SEQ ID NO: 66)
MNIIENIEKNDFSEIELAAIPFNTLADHYGSALAREQLALEHESYELGER
RFLKMLERQAKAGEIADNAAAKPLLATLLPKLTTRIVEWLEEYATKKGRK
PVAYAPLQSLKPEASAFITLKVILASLTSTNMTTIQAAAGMLGKAIEDEA
RFGRIRDLEAKHFKKHVEEQLNKRHGQVYKKAFMQVVEADMIGRGLLGGE
AWSSWDKETTMHVGIRLIEMLIESTGLVELQRHNAGNAGSDHEALQLAQE
YVDVLAKRAGALAGISPMFQPCVVPPKPWVAITGGGYWANGRRPLALVRT
HSKKGLMRYEDVYMPEVYKAVNIAQNTAWKINKKVLAVVNEIVNWKNCPV
ADIPSLERQELPPKPDDIDTNEAALKEWKKAAAGIYRLDKARVSRRISLE
FMLEQANKFASKKAIWFPYNMDWRGRVYAVPMFNPQGNDMTKGLLTLAKG
KPIGEEGFYWLKIHGANCAGVDKVPFPERIAFIEKHVDDILACAKDPINN
TWWAEQDSPFCFLAFCFEYAGVAHHGLSYNCSLPLAFDGSCSGIQHFSAM
LRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQ
VLDDTIQPAIDSGKGLMFTQPNQAAGYMAKLIWDAVSVTVVAAVEAMNWL
KSAAKLLAAEVKDKKTKEILRHRCAVHWTTPDGFPVWQEYRKPLQKRLDM
IFLGQFRLQPTINTLKDSGIDAHKQESGIAPNFVHSQDGSHLRMTVVYAH
ENYGIESFALIHDSFGTIPADAGKLFKAVRETMVITYENNDVLADFYDQF
ADQLHETQLDKMPPLPKKGNLNLQDILKSDFAFA
Enterobacteria bacteriophage T7M
(SEQ ID NO: 67)
MNIIENIEKNDFSEIELAAIPFNTLADHYGSALAKEQLALEHESYELGER
RFLKMLERQAKAGEIADNAAAKPLLATLLPKLTTRIVEWLEEYASKKGRK
PSAYAPLQLLKPEASAFITLKVILASLTSTNMTTIQAAAGMLGKAIEDEA
RFGRIRDLEAKHFKKHVEEQLNKRHGQVYKKAFMQVVEADMIGRGLLGGE
AWSSWDKETTMHVGIRLIEMLIESTGLVELQRHNAGNAGSDHEALQLAQE
YVDVLAKRAGALAGISPMFQPCVVPPKPWVAITGGGYWANGRRPLALVRT
HSKKGLMRYEDVYMPEVYKAVNLAQNTAWKINKKVLAVVNEIVNWKNCPV
ADIPSLERQELPPKPDDIDTNEAALKEWKKAAAGIYRLDKARVSRRISLE
FMLEQANKFASKKAIWFPYNMDWRGRVYAVPMFNPQGNDMTKGLLTLAKG
KPIGEEGFYWLKIHGANCAGVDKVPFPERIAFIEKHVDDILACAKDPINN
TWWAEQDSPFCFLAFCFEYAGVTHHGLSYNCSLPLAFDGSCSGIQHFSAM
LRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQ
VLDDTIQPAIDSGKGLMFTQPNQAAGYMAKLIWDAVSVTVVAAVEAMNWL
KSAAKLLAAEVKDKKTKEILRHRCAVHWTTPDGFPVWQEYRKPLQKRLDM
IFLGQFRLQPTINTLKDSGIDAHKQESGIAPNFVHSQDGSHLRMTVVYAH
EKYGIESFALIHDSFGTIPADAGKLFKAVRETMVITYENNDVLADFYSQF
ADQLHETQLDKMPPLPKKGNLNLQDILKSDFAFA
Enterobacteria bacteriophage T3
(SEQ ID NO: 68)
MNIIENIEKNDFSEIELAAIPFNTLADHYGSALAKEQLALEHESYELGER
RFLKMLERQAKAGEIADNAAAKPLLATLLPKLTTRIVEWLEEYASKKGRK
PSAYAPLQLLKPEASAFITLKVILASLTSTNMTTIQAAAGMLGKAIEDEA
RFGRIRDLEAKHFKKHVEEQLNKRHGQVYKKAFMQVVEADMIGRGLLGGE
AWSSWDKETTMHVGIRLIEMLIESTGLVELQRHNAGNAGSDHEALQLAQE
YVDVLAKRAGALAGISPMFQPCVVPPKPWVAITGGGYWANGRRPLALVRT
HSKKGLMRYEDVYMPEVYKAVNLAQNTAWKINKKVLAVVNEIVNWKNCPV
ADIPSLERQELPPKPDDIDTNEAALKEWKKAAAGIYRLDKARVSRRISLE
FMLEQANKFASKKAIWFPYNMDWRGRVYAVPMFNPQGNDMTKGLLTLAKG
KPIGEEGFYWLKIHGANCAGVDKVPFPERIAFIEKHVDDILACAKDPINN
TWWAEQDSPFCFLAFCFEYAGVTHHGLSYNCSLPLAFDGSCSGIQHFSAM
LRDEVGGRAVNLLPSETVQDIYGIVAQKVNEILKQDAINGTPNEMITVTD
KDTGEISEKLKLGTSTLAQQWLAYGVTRSVTKRSVMTLAYGSKEFGFRQQ
VLDDTIQPAIDSGKGLMFTQPNQAAGYMAKLIWDAVSVTVVAAVEAMNWL
KSAAKLLAAEVKDKKTKEILRHRCAVHWTTPDGFPVWQEYRKPLQKRLDM
IFLGQFRLQPTINTLKDSGIDAHKQESGIAPNFVHSQDGSHLRMTVVYAH
EKYGIESFALIHDSFGTIPADAGKLFKAVRETMVITYENNDVLADFYSQF
ADQLHETQLDKMPPLPKKGNLNLQDILKSDFAFA
Phage RNA polymerase
(SEQ ID NO: 69)
MNIINIAKNDFSDIELAAIPFNILADHYGAQLAREQLALEHEAYEEGEKR
FLKMLERQIKAGEFADNAAAKPLLSTLLPKLIARINDWFEEVAAKRGKKP
VAYNPLQHVKPEAAAFITLKVTLACLTKAEFTTIQAVASAIGRAIEDEAR
FGRIRDLEAKHFKKHVEEQLNKRVGHVYKKAFMQVVEADMLSKGLLGGEA
WSSWTKEESIHVGVRMLELLIESTGLVELHRPNAGNVGKDVEMIQLAPEY
VDLLAKRAGALAGISPMYQPCVVPPKPWTSIVGGGYWANGRRPLALVRTH
SKKALMRYEDVYMPEVYKAVNIAQNTPWKINKKVLAVVNEIVNWKHCPVA
DVPAIEREELPPKPEDIDTNEAALKAWKKAAAAIYRKDKARVSRRLSMEF
MLEQANKFANFKAIWFPYNMDWRGRVYAVPMFNPQGNDMTKGLLTLAKGK
PIGKDGFYWLKIHGANCAGVDKVPFPERIKFIEDNHENIMACAKDPLNNE
WWAEQDSPFCFLAFCFEYAGVQHHGLSYNCSLPLAFDGSCSGIQHFSAML
RDEIGGRAVNLLPSETVQDIYGIVADKVNEILKQDAINGTDNEVETVTDK
DTGEITEKLKLGTKELAGQWLAYGVTRKVTKRSVMTLAYGSKEFGFRQQV
LEDTIQPAIDSGKGLMFTQPNQAAGYMAKLIWEAVSVTVVAAVEAMNWLK
SAAKLLAAEVKDKKTKEVLRKRCAVHWVTPDGFPVWQEYRKPVQTRLNLM
FLGQFRLQPTINTNKDSEIDAHKQESGIAPNFVHSQDGSHLRMTVVHAHE
KYGIESFALIHDSFGTIPADAGNLFKAVRETMVNTYEDNDVLADFYDQFA
DQLHESQLDKMPALPAKGNLNLQDILKSDFAFA
Phage RNA polymerase
(SEQ ID No: 70)
MNTINIAKNDFSDIELAAIPFNTLADHYGERLAREQLALEHESYEMGEKR
FLKMLERQVKAGEIADNAAAKPLITTLLPKLTARINDWFEEVAAKRGKRP
VAYQPLQGIKPEAVAFITIKVVLASLTSADNTTIQAVASAIGRAIEDEAR
FGRIRDLEAKHFKKHVEEQLNKRVGHVYKKAFMQVVEADMLSKGLLGGEA
WSSWNKEESMHVGIRMIEMLIESTGLVELHRHNAGVVGQDSETIQLAPEY
VEALAKRAGALAGISPMFQPCVVPPKPWVSITGGGYWANGRRPLALVRTH
SKKALMRYEDVYMPEVYKAVNIAQNTAWKINKKVLAVVNEIVNWKHCPVE
DIPAIEREELPPKPDDIDTNEEALKAWKKAAAAVYRKDKARKSRRISLEF
MLEQANKFANHKAIWFPYNMDWRGRVYAVPMFNPQGNDMTKGLLTLAKGK
PIGKEGFYWLKIHGANCAGVDKVPFPERIKFIEDNHDNIMACAKDPLDNT
WWAEQDSPFCFLAFCFEYAGVQHHGLSYNCSLPLAFDGSCSGIQHFSAML
RDEVGGRAVNLLPSETVQDIYGIVADKVNEILKQDVINGTDNEVVTVTDK
DTGEISEKLKLGTKELAQQWLAYGVTRSVTKRSVMTLAYGSKEFGFRQQV
LEDTIQPAIDSGKGLMFTQPNQAAGYMAKLIWDAVSVTVVAAVEAMNWLK
SAAKLLAAEVKDKKTKEILRKRCAVHWVTPDGFPVWQEYRKPIQTRLNLM
FLGQFRLQPTINTNKDSEIDAHKQESGIAPNFVHSQDGSHLRMTVVYAHE
KYGIESFALIHDSFGTIPADAGNLFKAVRETMVNTYENNDVLADFYDQFA
DQLHESQLDKMPALPAKGNLNLQDILKSDFAFA

### EXAMPLES

### Example 1: Initial Screening Assays

Structure-based calculations were carried out to predict effect of mutations on thermal stability of T7 RNA polymerase. Mutations were modeled and evaluated using BioLuminate™ software (Schrödinger, New York, N.Y.) starting with the crystal structure of the wild-type T7 RNA polymerase (PDB ID: 1MSW). The predicted change in protein thermal stability upon mutation (MG) was used to choose the candidate mutations. Mutations predicted to be stabilizing were introduced into wild type T7 RNA polymerase using site-directed mutagenesis using the Q5® Site-Directed Mutagenesis Kit (New England Biolabs, Ipswich, Mass.) and manufacturer's recommended protocols.

Individual mutations (see for example those in FIGS. 1A-1D) were screened in a novel cell-free assay based on the reconstituted translation system from Thermus thermophilus (Tth PURE system). Reconstitution of translation from Thermus thermophilus reveals a minimal set of components sufficient for protein synthesis at high temperatures and functional conservation of modern and ancient translation components (Zhou, et al., Nucleic Acids Research, 40(16), 7932-7945 (2012)). Genes encoding T7 RNAP variants were transcribed in vitro using SP6 RNA polymerase. 1 μl of in vitro transcription reaction was added to 10 μl of Tth PURE system with a fluorescent reporter gene (a GFP variant under the control of a T7 RNAP promoter). The activity of T7 RNA polymerase variants synthesized in Tth PURE system was coupled to the expression of a GFP gene under the control of a T7 promoter. All reactions were incubated at a range of temperatures for 10 hours. Transcription was monitored by production of a fluorescent signal in real time using a CFX96 Touch™ Real-Time PCR Detection System (Bio-Rad, Hercules, Calif.). FIG. 1A-1C show data for selected individual variants incubated at 45° C. for 10 hours (FIGS. 1A and 1B) and at 37° C. for 2 hours followed by 45° C. for 8 hours (FIG. 1C). FIG. 1D shows data for selected combinations of mutations. The reaction was carried out at 45° C. for 10 hours. All variants shown have a detectable increase in thermostability.

### Example 2: Melting Temperature and Temperature Dependence Analysis

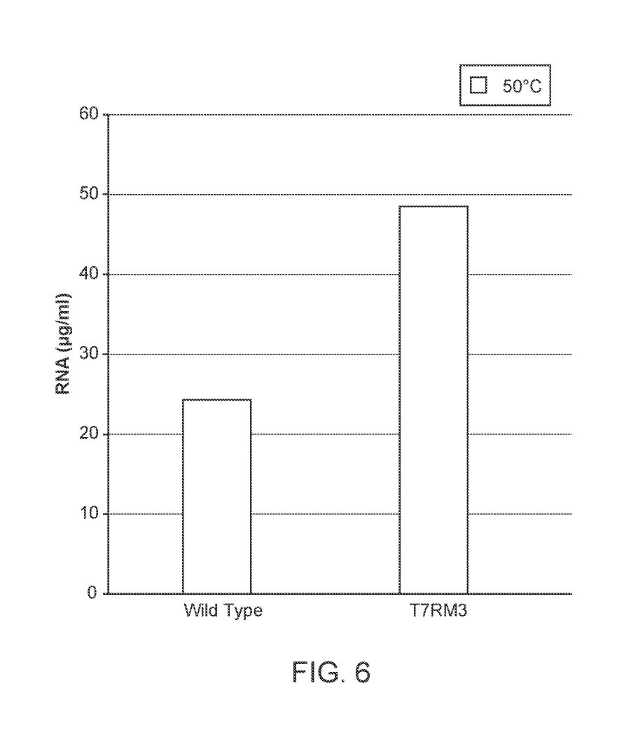

A. Selected T7 RNA polymerase variants (including those described in FIGS. 3A-3C, and FIGS. 4-6) contained an N-terminal hexahistidine tag and were expressed in E. coli and purified using nickel affinity chromatography using an ÄKTAFPLC® system (GE Life Sciences, Marlborough, Mass.). The hexahistidine-tagged polymerase variants were isolated and purified on nickel resin, eluted from the nickel resin with imidazole and dialyzed into a storage buffer (for example: 50 mM Tris-HCl pH 7.5, 100 mM NaCl, 10 mM DTT, 50% Glycerol, 0.1% Triton X-100).

B. To measure the melting temperature of the mutants (as shown in FIG. 2), 0.2 mg/ml T7 RNA polymerase variants were prepared in a buffer (50 mM Hepes-KOH, pH 8.0, 10 mM Mg(OAc)₂, 5 mM DTT, 2 mM spermidine). Melting temperatures were measured using Prometheus NT.48 (NanoTemper Technologies).

C. To determine the reaction temperature range (as shown in FIG. 3A), the yield of RNA synthesis was measured from 37° C. to 60° C. Each 25 μl reaction contains a final concentration of 50 mM Hepes-KOH, pH 7.5, 10 mM Mg(OAc)₂, 5 mM DTT, 2 mM spermidine, 1 mM NTP, 4 ng/μl linear DNA template of the Green Fluorescent protein reporter gene with T7 promoter, and 8 μg/ml T7 RNA polymerase variant. Reactions were run at various temperatures using Bio-Rad T100™ Thermal Cycler (Bio-Rad, Hercules, Calif.) for 2 hours. After the transcription reactions, 1 unit of DNase I (New England Biolabs, Ipswich, Mass.) was added and the reactions were incubated at 37° C. for 30 minutes.

Total synthesized RNA was measured using a Qubit® RNA BR Assay Kit (Thermo Fisher Scientific, Waltham, Mass.) to measure GFP mRNA.

### Example 3: Beacon Assays

Wild type T7 RNA polymerase and two different variants were fused to the sso7d DNA binding domain (of sequence ATVKFKYKGEEKEVDISKIKKVWRVGKMISFTYDEGGDKTGRGAVSEKDAPKELLQMLEKQKK; SEQ ID NO:6), the DNA binding domain from a helix-turn-helix (HTH) from Pyrococcus furiosus (of sequence GRKVRTQQNEILNLLNEKEKAVLRAILEHGGEIKQED LPELVGYSRPTISKVIQELENKGLIKREKSGKTFVVKIERKIKLDKMGAPT; SEQ ID NO:7) or the DNA binding domain of a lacl-like protein from Thermotoga (of sequence KRRPTINDVAKLAGVSISTVSRYLKDPSQVSEKLGERIREAIKKLGYKPNKIAQGLRTGD; SEQ ID NO:8). The fusion proteins were purified as described in Example 2A above. The fusion proteins were tested in a molecular beacon assay at various temperatures, as shown in FIGS. 3B-3C, FIG. 4 and FIG. 5 and their thermostability compared to variant RNA polymerases that were not fused. M13, M18 and M20 variants in FIGS. 3B-3C, FIG. 4 and FIG. 5 are examples of an amino acid sequence that is at least 80% sequence identity to SEQ ID NO:1; and include an amino acid substitution at positions corresponding to 109L, 205S, 388E, 534V, 567P and 618Q of SEQ ID NO:1.

The yields were compared using a molecular beacon assay that monitors in vitro transcription of a 6 Kb transcript. The in vitro transcription reactions were performed in 41 mM Tris-HCl pH 8.0, 50 mM NaCl, 19 mM MgCl₂, 5.5 mM DTT, 1 mM spermidine, 4 mM of each ribonucleotide, 4.15 units/mL yeast inorganic pyrophosphatase, 1000 units/mL murine ribonuclease inhibitor, 30 nM DNA template, 30 nM RNA polymerase, and 0.5 μM molecular beacon probe. A linearized plasmid DNA was used as template for the in vitro transcription reactions. The molecular beacon was designed to bind a 24 nucleotide target site upstream of the linearization site of the plasmid with a sequence of: 5′-CCT GC GATT GAA CAC GTG GGT CAG AGA GG GCAGG-3′ (SEQ ID NO:9). The molecular beacons were labeled with the fluorescent dye TYE665 at the 5′ end and the quencher IAbRQSp at the 3′ end (or with the fluorescent dye 6-FAM at the 5′ end and the quencher BHQ1 at the 3′ end) (Integrated DNA Technologies, Coralville, Iowa). Reactions were run at various temperatures using a CFX96 Touch Real-Time PCT Detection System for one hour. The graph denotes end-point fluorescence units (representing the final yield from the in vitro transcription reaction) obtained for each polymerase plotted against the temperature at which the reactions were run.

What is claimed is:

1. An isolated bacteriophage RNA polymerase variant, wherein the variant:
(i) comprises an amino acid sequence is at least 80% sequence identity to SEQ ID NO:1; and
(ii) comprises an amino acid substitution at one or more positions corresponding to positions selected from 109, 205, 388, 534, 567 and 618 of SEQ ID NO:1.

2. The isolated bacteriophage RNA polymerase variant of claim 1, wherein the variant further comprises an amino acid substitution of at least two positions corresponding to positions selected from 109, 205, 388, 534, 567 and 618 of SEQ ID NO:1.

3. The isolated bacteriophage RNA polymerase variant of claim 1, wherein the variant comprises an amino acid substitution of at least three positions corresponding to positions selected from 109, 205, 534 and 618 of SEQ ID NO:1.

4. The isolated bacteriophage RNA polymerase variant of claim 1, wherein the variant further comprises an amino acid substitution at positions corresponding to positions 109, 205, 534 and 618 of SEQ ID NO:1.

5. The isolated bacteriophage RNA polymerase variant of claim 1, wherein the variant comprises one or more of the following amino acids substitutions selected from I109L, H205S, D388E, L534V, V567P and G618Q wherein the amino acid substitutions are at positions that correspond to positions in SEQ ID NO:1.

6. The isolated bacteriophage RNA polymerase variant of claim 1, wherein the variant further comprises an amino acid substitution at one or more positions corresponding to positions selected from: 75, 83, 108, 206, 227, 281, 297, 312, 323, 327, 333, 340, 354, 362, 375, 428, 446, 454, 461, 495, 510, 584, 591, 642, 711, 724, 740, 788, 832, 834, 835, 843, 847, 849, 856, 863, 866 and 877 of SEQ ID NO:1.

7. The isolated bacteriophage RNA polymerase variant of claim 1, wherein the variant further comprises an amino acid substitution of at least 10 positions corresponding to positions selected from: 75, 83, 108, 206, 227, 281, 297, 312, 323, 327, 333, 340, 354, 362, 375, 428, 446, 454, 461, 495, 510, 584, 591, 642, 711, 724, 740, 788, 832, 834, 835, 843, 847, 849, 856, 863, 866 and 877 of SEQ ID NO:1.

8. The isolated bacteriophage RNA polymerase variant of claim 1, wherein the variant further comprises one or more of the following amino acids substitutions selected from: T75Q, A83K, E108L, K206P, V227I, I281P, V297I, Y312D, A323I, A327P, K333P, V340E, A354Q, M362P, T375K, T375N, A428P, L446F, K454P, K461R, S495N, C510Q, A584K, D591E, K642R, K711R, A724P, K740R, G788A, M832F, D834E, T835L, A843Q, D847E, F849V, S856T, A863P, A866K and E877R, wherein the amino acid substitutions are at positions that correspond to positions in SEQ ID NO:1.

9. The isolated bacteriophage RNA polymerase variant of claim 1, wherein the variant further comprises at least 10 of the following amino acids substitutions selected from: T75Q, A83K, E108L, K206P, V227I, I281P, V297I, Y312D, A323I, A327P, K333P, V340E, A354Q, M362P, T375K, T375N, A428P, L446F, K454P, K461R, S495N, C510Q, A584K, D591E, K642R, K711R, A724P, K740R, G788A, M832F, D834E, T835L, A843Q, D847E, F849V, S856T, A863P, A866K and E877R wherein the amino acid substitutions are at positions that correspond to positions in SEQ ID NO:1.

10. The isolated bacteriophage RNA polymerase variant of claim 1, wherein the variant comprises a fusion to an exogenous DNA binding domain.

11. The isolated bacteriophage RNA polymerase variant of claim 1, wherein, as a result of the one or more amino acid substitutions, the variant has increased stability at temperatures above 45° C., 50° C. or 55° C. relative the T7 RNA polymerase of SEQ ID NO:1.

12. The isolated bacteriophage RNA polymerase variant of claim 1, wherein, as a result of the one or more amino acid substitutions, the variant has increased activity at temperatures above 42° C., 45° C., 50° C. or 55° C. relative to the T7 RNA polymerase of SEQ ID NO:1.

13. The isolated bacteriophage RNA polymerase variant of claim 1, comprising an amino acid sequence that is at least 90% sequence identity to SEQ ID NO:1.

14. The isolated bacteriophage RNA polymerase variant of claim 1, comprising an amino acid sequence that is at least 90% sequence identity to any of SEQ ID NO:52-70.

15. A composition comprising:
(i.) an isolated bacteriophage RNA polymerase variant of claim 1; and
(ii.) a buffering agent.

16. The composition of claim 15, further comprising ribonucleoside triphosphates and/or a modified nucleotide.

17. The composition of claim 15, further comprising a template DNA molecule comprising: a promoter, for example, a bacteriophage RNA polymerase promoter, operably linked to a target nucleotide sequence to be transcribed.

18. A kit comprising:
(i) an isolated bacteriophage RNA polymerase variant of claim 1; and
(ii) a reaction buffer.

19. The kit of claim 18, wherein the kit further comprises one or more ribonucleoside triphosphates.

20. A method for synthesizing an RNA molecule comprising:
(a) combining an isolated bacteriophage RNA polymerase variant of claim 1, with ribonucleoside triphosphates and/or a modified nucleotide and a template DNA molecule comprising a bacteriophage RNA polymerase promoter that is operably linked to a target nucleotide sequence to be transcribed, to produce a reaction mix; and
(b) incubating the reaction mix to transcribe the template DNA molecule into RNA.

21. The method of claim 20, wherein the incubating is done at a temperature of at least 45° C.

22. The method according to claim 20, wherein the bacteriophage RNA polymerase is T7 RNA polymerase.

23. The method of claim 20, wherein the bacteriophage RNA polymerase is T7 RNA polymerase.

24. The method of claim 21, wherein the method is a Nucleic Acid Sequence Based amplification (NASBA) method that comprises: reverse transcribing an RNA template to produce an RNA-cDNA hybrid, treating the RNA-cDNA hybrid to destroy the RNA template and produce a DNA strand, hybridizing a primer to the DNA strand, wherein the primer comprises the bacteriophage RNA polymerase promoter, extending the primer to produce a second strand, and transcribing the second strand using the bacteriophage RNA polymerase to produce an RNA product.

25. The method of claim 21, further comprising detecting the RNA product.
